(19) United States
(12) Patent Application Publication
(10) Pub. No.: US 2018/0358130 A1
(43) Pub. Date: Dec. 13, 2018

(54) SYSTEMS AND METHODS FOR USING AGGREGATE COMMUNITY HEALTH STATISTICS IN CONNECTION WITH DISEASE PREVENTION PROGRAMS

(71) Applicant: Brenda Schmidt, Phoenix, AZ (US)

(72) Inventor: Brenda Schmidt, Phoenix, AZ (US)

(21) Appl. No.: 16/106,909

(22) Filed: Aug. 21, 2018

Related U.S. Application Data
(63) Continuation-in-part of application No. 14/808,956, filed on Jul. 24, 2015.

(51) Int. Cl. G16H 50/70; G06Q 40/08; G06Q 20/10; G16H 10/60

(52) U.S. Cl. CPC G16H 50/70; G06Q 40/08; G06T 11/206; G16H 10/60; G06Q 20/10

(57) ABSTRACT

Systems and methods are provided for integrating consumer data with geospatial data relating to disease metrics to generate and deliver custom interventions for personalized health plans. Aggregate community health statistics are used to drive patient enrollment in disease prevention programs.

### CROSS-REFERENCES TO RELATED APPLICATIONS

This patent application is a continuation-in-part of U.S. patent application Ser. No. 14/808,956, filed Jul. 24, 2015, which is incorporated by reference herein.

### TECHNICAL FIELD

The present invention relates, generally, to healthcare services and, more particularly, to removing barriers to healthcare for particular subsets of the population.

### BACKGROUND

The evolving U.S. health care system presents opportunities for improving population health. A key component of population health involves linking clinical care with community-based prevention programs and related social services. Shifting the emphasis to embracing population-based health principles can have a greater effect on long term health and wellness, particularly in the prevention of chronic disease.

Under various statutory schemes, non-profit health plans and hospitals are required to develop a Community Health Needs Assessment (“CHNA”), including a quality improvement analysis tool to substantiate improvements in community health care in order to maintain their non-profit status. When conducted systematically, the CHNA process can be a driver to implement evidence-based strategic interventions that address prioritized community health needs. This is one step in improving the health of communities.

Currently, the Brief Risk Factor Surveillance System (“BRFS”), which is maintained by the Center for Disease Control (“CDC”), collects data about U.S. residents regarding their health-related risk behaviors, chronic health conditions, and use of preventive services. However, these data are typically maintained at the county or state level, which is inadequate to drive decisions at a more granular level, such as zip code or neighborhood.

The widespread interest in the role of social health determinants has renewed emphasis on implementing interventions to improve socioenvironmental conditions. Such interventions have the potential to produce wide-ranging health benefits and could reduce marked health disparities that remain a high-priority for community health. This recent interest has heightened the need for improved conceptual data on how the social environment impacts the health of populations.

Systems and methods are thus needed which overcome these and other limitations of the prior art. Various desirable features and characteristics will also become apparent from the subsequent detailed description and the appended claims, taken in conjunction with the accompanying drawings and this background section.

### BRIEF SUMMARY

Various embodiments of the present invention relate to systems and methods for, inter alia, (i) integrating community based disease prevention program (“DPP”) data with BRFS and other geographical data on disease metrics to generate a dashboard driven by business intelligence rules to thereby present a health plan administrator and/or an integrator with graphical and/or textual summaries for the efficient design of intervention policies at the zip code or neighborhood level; (ii) mapping the results onto a community dashboard, which can be used for developing, implementing, and monitoring a personalized prevention and management plan based on available community resources; and (iii) providing a graphical user interface for a database including a “heat map,” with a resolution to at least a zip code level, illustrating chronic diseases, social determinants of health, and community health care providers associated with distinct geographic areas.

Various other embodiments, aspects and features are described in greater detail below.

### BRIEF DESCRIPTION OF THE DRAWING FIGURES

Exemplary embodiments will hereinafter be described in conjunction with the following drawing figures, wherein:

FIG. 1 is a schematic block diagram of an exemplary integrator-centric system for facilitating the provision of disease prevention programs, in accordance with various embodiments;

FIG. 2 is a schematic block diagram of an exemplary system for implementing a marketplace, which includes a consumer, an integrator, a payer, a social support provider, and a disease prevention program provider, in accordance with various embodiments;

FIG. 3 is a process flow diagram illustrating an exemplary use case involving a consumer, a payer, a disease prevention program provider, a social support provider, and an integrator, in accordance with various embodiments;

FIG. 4 is an alternative process flow diagram illustrating an exemplary use case involving a consumer, a payer, a disease prevention program provider, a social support provider, a behavioral health provider, and an integrator, in accordance with various embodiments;

FIG. 5 is a flow chart illustrating exemplary steps for determining whether a consumer is in need of a social resource to increase a likelihood of success in a disease prevention program, in accordance with various embodiments;

FIG. 6 is a flow chart illustrating exemplary steps for enrolling a consumer into a disease prevention program, in accordance with various embodiments;

FIG. 7 is a flow chart illustrating exemplary steps for analyzing data to determine a market for one or more disease prevention programs in a location, in accordance with various embodiments;

FIG. 8 is a schematic block diagram of an exemplary integrator system configured to generate a personalized precision prevention plan for three exemplary participants, in accordance with various embodiments; and

FIG. 9 illustrates an example heat map in accordance with various embodiments.

The drawings described herein are for illustrative purposes only of selected embodiments and not all possible implementations, and are not intended to limit the scope of any of the exemplary embodiments disclosed herein or any equivalents thereof.

### DETAILED DESCRIPTION

The following detailed description of the invention is merely exemplary in nature and is not intended to limit the invention or the application and uses of the invention. Furthermore, there is no intention to be bound by any theory presented in the preceding background or the following detailed description.

Various embodiments of the present invention relate to systems and methods for linking primary care providers with community-based organizations (“CBOs”) or virtual providers to provide disease prevention and other related programs. The present invention further contemplates systems and methods for applying business logic to zip code level health data to facilitate custom health programs for individual participants based on available CBO resources. The health programs may include, for example, the following categories: i) lifestyle pre-chronic prevention; ii) chronic disease; iii) behavioral health; and iv) pharmaceutical compliance and dosage protocols.

In accordance with one embodiment, a database is configured to map (with resolution to at least a zip code level and sometimes even down to the neighborhood level) chronic diseases, social determinants of health (e.g., public transportation), and community health care providers to distinct geographic areas. An integrator can build an API between this “zip code” database and the integrator system, and the integrator can then apply business intelligence to make the zip code database actionable by recommending policies and interventions calculated to positively impact health care costs and quality. More particularly, the business intelligence may be applied to the zip code database to assist an integrator in developing personalized prevention and management plans based on available community resources. Based on this information, recommendations for objectively improving health care metrics can be implemented for an individual participant.

In accordance with some embodiments, a community dashboard is configured to create and monitor a personalized prevention and management plan based on available community resources. This dashboard can be configured to receive the raw data from a geospatial aggregate health database and render it actionable for health plan providers. In some implementations, the geospatial aggregate health database is configured as a spatial data infrastructure designed to acquire data, process it, store results, and preserve any accompanying spatial data. In some implementations, the geospatial aggregate health database can be configured as a geographical information system (GIS).

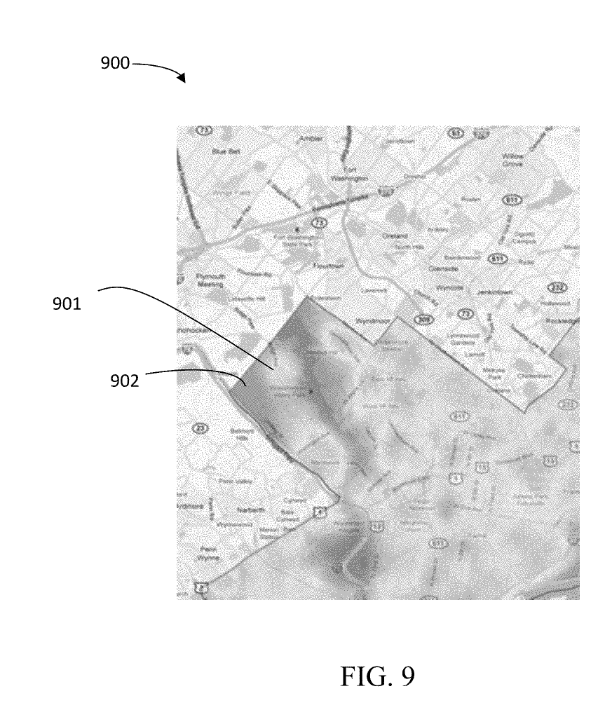

In one embodiment, the geospatial aggregate health database includes cultural factors, language, educational factors, economic factors, food insecurity, access to a vehicle, single parent status, crime statistics, child care, nutrition, disabilities, Medicare, and community assets such as pharmacies, clinics, hospitals. By inputting one or more search criteria, the system produces a heat map (i.e., a two-dimensional representation in which numerical values are mapped to colors or shades of a single color) of a defined location or area, which may be a zip code, a census block, or a neighborhood. Referring momentarily to FIG. 9, a heat map 900 generally includes a geographical area upon which is superimposed a translucent layer including, for example, areas 901 that are lighter (or a first color) and areas 902 that are darker (or a second color). The colors or lightness may be continuous or quantized into discrete values. The geographical area may include a variety of features and may be partitioned by zip code, neighborhood, or the like. The heat map allows the integrator to build out a network of providers specifically tuned to the needs of an individual participant, or to certain demographics within the defined location.

In one embodiment, the heat map is a representation of the resulting data on a map in which the data values are represented as colors ranging from green (lowest) to red (highest), such that “hot spots” of populations at risk are easily identified on the heat map. In some embodiments, an interactive heat map of the defined location is included on the dashboard.

In some embodiments, the integrator has the power to enroll people in certain programs, and is thus uniquely qualified to combine the geospatial aggregate health data with the individual patient profiles to determine the demand in a defined area of a market for one or more disease prevention programs. Based on this demand, the integrator can recruit and build out the program providers (such as CBOs) in a particular location, which allows the appropriate participants to be enrolled in the best-fit DPP in the location. Since the integrator has access to patient profile information, this allows the aggregate data to be actionable in a location, which can trigger can enrollment in a disease prevention program (DPP″) for a candidate in the location.

The geospatial aggregate health database may include data from many sources. In some configurations, the geospatial aggregate health database can include data licensed from a third party. For example, Blue Cross Blue Shield (“BCBS”) has reported that it has a database of the impact of 200 different diseases and condition on over 40 million members. The entries in the BCBS database can be sorted and tagged by location (such as, for example, zip code) for each of the 200 different diseases and conditions, thereby creating a geospatial layer for the heat map for each of the 200 different diseases and conditions.

In various embodiments, systems and methods integrate CBO provider data, including the nature and location of local community based resources, with BRFS and other geographical data on disease metrics to generate a dashboard driven by business intelligence rules to thereby present a health plan administrator and/or an integrator with graphical and/or textual summaries for the efficient design of intervention policies at the zip code or neighborhood level.

In some embodiments, the integrator partners with health plans and determines where to send the health plan patients in their neighborhood. The health plan pays the integrator to place the patient in a DPP class. In some configurations, the integrator bundles non-clinical services to address a social determinant (such as, an Uber ride) with clinical services (such as, a DPP) into one claim and the health plan pays the claim. In some embodiments, the system can perform a Community Health Needs Assessment for one or more non-profit healthcare providers.

According to the Center for Disease Control and Prevention (“CDC”), social determinants of health are factors in the social environment that contribute to or detract from the health of individuals and communities. These factors may include, but are not limited to, socioeconomic status, transportation, housing, access to services, discrimination by social grouping, and social or environmental stressors. In adding socioeconomic status to a heat map, several layers of data can be used, such as, for example, poverty level, unemployment, high school education, and levels of healthcare insurance. Any number of additional layers of socioeconomic data can be added to the heat map. These layers of data for the heat map can be stored in the geospatial aggregate health database. In some embodiments, social determinants of health include economic stability, education, health care, neighborhood and build environment, and social and community context.

Economic stability may include layers for poverty, employment, food insecurity, and housing instability. Education may include layers for high school graduation, enrollment in higher education, languages, literacy, and early childhood education and development. Health care may include layers for access to health care, access to primary care, and health literacy. Neighborhood and build environment may include layers for access to foods that support healthy eating patterns, quality of housing, crime and violence, environmental conditions, and transportation. Social and community context may include layers for social cohesion, languages culture, civic participation, discrimination, and incarceration.

Various embodiments provide systems and methods for comparing the health risk factors of a population to the capacity of the healthcare resources for the population and identifying areas of needs for additional resources for the community. Some embodiments provide systems and methods for targeting a demographic subset of the population in a defined area and removing barriers to healthcare for the subset of the population.

Using predictive analytics, an integrator system can determine a profile of the “ideal participant” for each program provider based on delivery methods and individual participant characteristics. This profile represents a hypothetical participant most likely to be successful in each program based on a delivery methodology.

After matching participant-specific data to various ideal participant profiles, the system programmatically (e.g., algorithmically) selects the program provider best suited to the participant. In this context, the participant-specific data may include, for example, patient contact information (including zip code), demographics, socio-economic factors, social determinants, psychographics, health information, health care utilization, claims data, electronic medical record data, prescription history, and purchasing data.

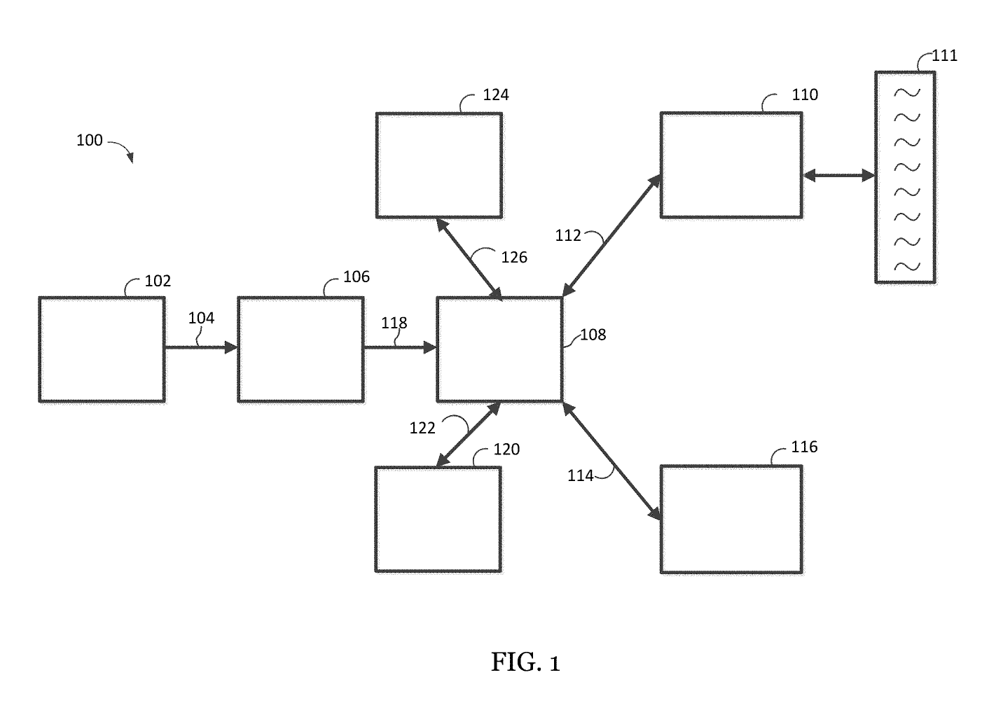

More particularly and referring now to FIG. 1, an exemplary integrator-centric system loo for delivering at least one DPP 110 will now be described. In general, a clinical provider 102 is configured to refer 104 a candidate 106 to an integrator 108. The clinical provider 102 may include, but is not limited to, a doctor or a hospital. The referral 104 may be in the form of a professional referral, a prescription, or the like. The candidate 106 may correspond, for example, to a patient, a client of a medical service, a program participant, a consumer, and/or a user.

In some embodiments, the integrator 108 has access to at least one third-party database 120 containing information about potential candidates, from which the integrator 108 identifies the candidate 106. The integrator 108 communicates 118 with the candidate 106, e.g., to determine whether the candidate 106 is eligible for a program. The integrator 108 can invite the candidate 106 to join a DPP 110 and enroll the candidate 106 in a DPP 110.

The third-party database 120 is configured to provide data 122 to integrator 108. The data 122 may be for a specific group, or may be for a population located in a defined area, or both. The integrator 108 can parse through the data 122 to identify potential candidates for a DPP 110 located in the specific area.

In some aspects, the database 120 can be created by analyzing and aggregating a large database of claims data. The integrator 108 can use the data 122 to generate heat maps illustrating which areas have a higher incidence of certain treatable conditions. The data 122 can be used by the integrator 108 to plan on what programs, DPPs 110, social services, and/or CBOs need to be built in a specific area. The data base 120 can include consumer information, which the integrator 108 can use to contact and enroll one or more potential candidates identified in the heat map into an appropriate program designed for the prevention of a certain treatable condition.

The database 120 can be configured to store raw data from a geospatial aggregate health database, and provide data 122, which is actionable by the integrator 108. In some implementations, the geospatial aggregate health database 120 can be configured as a spatial data infrastructure designed to acquire data, process it, and store results, and preserve any accompanying spatial data. In some implementations, the geospatial aggregate health database 120 can be configured as a geographical information system (GIS). In some implementations, the database 120 can comprise BRFS and other geographical data on disease metrics at zip code or neighborhood level.

In some embodiments, the database 120 is a geospatial aggregate health database configured to catalogue data describing one or more social determinants, such as cultural factors, language, educational factors, economic factors, food insecurity, access to a vehicle, single parent status, crime statistics, child care, nutrition, disabilities, Medicare, and community assets, such as pharmacies, clinics, hospitals DPP providers, and CBOs. The integrator 108 may enter one or more search criteria into the database 120, such as, for example “Spanish language,” “diabetes,” or “cardiovascular disease”—to produce data 122 for a specific population subset in a defined area. The integrator 108 can then use the data 122 to produce a heat map of the subset for the defined area. The results of the heat map can be compared to database in of DPP providers for the defined area to determine if the capacity of DPP providers meets the needs (health and and/or social) of the population subset in the defined area. Based on this knowledge, the integrator 108 can build a network of DPP providers specifically tuned to the needs of the population subset within the defined location.

The candidate 106 communicates 118 with integrator 108 by entering data responsive to a health and lifestyle survey. This survey data can be analyzed by a patient health risk stratification system, which is configured to recognize more than one chronic disease and configured to determine the highest priority chronic diseases for the candidate 106. A list of the highest priority chronic diseases can be used match a corresponding DPP 110 for each chronic disease on the list.

However, the process of matching a candidate 106 to a best-fit DPP no involves determining whether the candidate 106 is eligible for one or more DPPs 110 based on objective criteria, as defined by the candidate's payer 116. For example, a candidate 106 with a wellness score in the low risk level may not be eligible for benefits covering a DPP 110. However, the candidate 106 can be identified as eligible by integrator 108 through the data 122 provided from a search of the database 120.

If the candidate 106 is eligible for any of the corresponding DPP 110 for each chronic disease on the list, the candidate 106 inputs data responsive to a personal profile survey.

The integrator 108 accesses a database 111 of DPP providers and recommends a “best-fit” DPP 110 based on, for example, correlation between the personal profile entered by the candidate 106 and one of a plurality of ideal participant profiles, each associated with a DPP provider. The integrator 108 enrolls the candidate 106 into the best-fit DPP 110.

A key component of population health involves linking clinical care with community-based prevention programs and related social services. For example, the candidate 106 can have one or more social needs, in addition to a need for a DPP 110. In some configurations, the integrator 108 bundles non-clinical services, (for example, cost of transportation) with clinical services (for example, a DPP 110) into one claim. The payer 116 receives the claim and sends payment 114 to the integrator 108 for both services. The integrator 108 can distribute this payment 114 to both the social support provider (for example, for the cost of transportation) and the DPP provider (for example, for providing the DPP).

The system 100 may include a social support provider database 124, which can used to identify a “best-fit” social service provider, as a result of a query using a search criteria (including the social success needs) provided by the integrator 108. In some implementations of the system boo, the search criteria provided by the integrator 108 is based on the candidate's survey data in response to a health and lifestyle survey. In some implementations of the system 100, the search criteria is based on the data 122 from a query of the database 120 and a resulting heat map of a targeted population subset for a defined area. In some implementations of the system 100, the search criteria is based on both the candidate's survey data and the heat map generated from the data 122.

In one example, a heat map includes a layer for access to public transportation. A candidate 106 is enrolled in DPP 110 within the boundaries of the heat map. The heat map can be configured to determine whether the candidate's residence is located in an area serviced by public transportation. If the candidate's residence is outside the area serviced by public transportation, the integrator 108 can query the social provider database 124 for a social solution 126 for transporting the candidate 106 to the DPP 110, such as, a community van, a taxi cab, or a ride share. However, if the candidate's survey data indicates that the candidate 106 is able to drive and has an operable car, and the analyzing the heat map for the area serviced by public transportation may not be required. In some aspects, the integrator 108 can analyze the candidate's survey data, and then determine if an analysis of the heat map is required.

The integrator 108 monitors 112 the candidate's compliance with the DPP 110 (and the use of the social solution 126), then processes a claim for payment 114 from a payer 116. The payer 116 may be an insurance company, Medicaid, Medicare, a health system, or health plan administrator. The integrator 108 may be configured to process a claim for payment 114 by performing one or more of the steps of: submitting a bundled claim to the payer 116, receiving approval for the bundled claim from the payer 116, invoicing the payer 116, and receiving the payment 114 for the bundled claim from the payer 116. The integrator 108 can send the initial claim to the payer 116 upon enrollment of the candidate 106 in the DPP 110. Upon receipt of the payment 114, the integrator 108 can send a portion of the payment 114 to the DPP provider and a portion of the payment 114 to the social support provider.

A computer system 100 is provided for using aggregate community health statistics to drive patient enrollment in disease prevention programs. In one embodiment, computer system 100 is configured to perform the steps of: analyzing a database comprising a plurality of community health statistics; generating a plurality of geospatial data layers of disease incidence from the plurality of community health statistics; creating a heat map comprising the plurality of geospatial data layers; segmenting the heat map into smaller defined areas; determining which of the defined areas have a high incidence of a preventable disease; analyzing a patient database comprising a plurality of patient names, each of the patient names tagged with a patient's address and the patient's contact information; identifying a group of patients in the defined areas with the high incidence of the preventable disease; contacting each of the identified patients in the group of patients; and enrolling at least a portion of the identified patients into a disease prevention program for the preventable disease.

The computer system loo may be configured to perform the steps of: surveying each of the identified patients for personal preferences for the disease prevent program; and determining a best-fit disease prevention program provider for each of the identified patients based on comparing the personal preferences.

In various embodiments, computer system 100 is configured to perform the steps of: submitting a claim for the disease prevention program for each of the identified patients to a group of payers; receiving a payment for at least one claim from the group of payer; and sending a portion of the payment to a disease prevention program provider.

In various embodiments, the computer system 100 is configured to perform the steps of: determining which of the defined areas have a high incidence of a second preventable disease; analyzing the patient database; identifying a second group of patients in the defined areas with the high incidence of the second preventable disease; contacting each of the identified patients in the second group of patients; and enrolling at least a portion of the identified patients in the second group of patients into a disease prevention program for the second preventable disease.

The computer system 100 may also be configured to perform the steps of: surveying each of the identified patients for social needs for success in the disease prevention program; determining a highest impact social need for increasing a likelihood of success in the disease prevention program for each of the identified patients; developing a best-fit social solution for the highest impact social need for each of the identified patients with a group of social support providers; determining a best-fit social support provider for each of the identified patients from the group of social support providers; and enrolling each of the identified patients in the best-fit social solution with the best-fit social support provider.

In some embodiments, computer code is stored in a non-transient medium for performing, when executed by a computer processor 100, the steps of: analyzing a database comprising a plurality of community health statistics; generating a plurality of geospatial data layers of disease incidence from the plurality of community health statistics; creating a heat map comprising the plurality of geospatial data layers; segmenting the heat map into smaller defined areas; determining which of the defined areas have a high incidence of a preventable disease; analyzing a patient database comprising a plurality of patient names, each of the patient names tagged with a patient's address and the patient's contact information; identifying a group of patients in the defined areas with the high incidence of the preventable disease; contacting each of the identified patients in the group of patients; and enrolling at least a portion of the identified patients into a disease prevention program for the preventable disease.

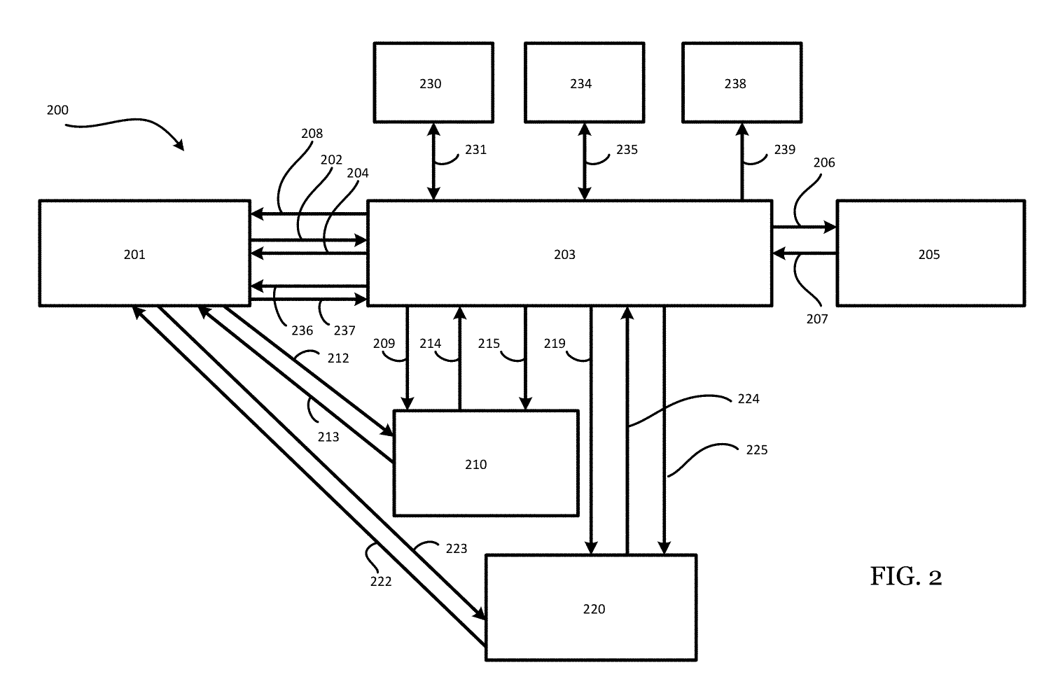

With reference to FIG. 2, an exemplary system 200 for implementing a three-sided marketplace metaphor includes an integrator 203, a consumer 201, a payer 205, a social support provider 220, and a DPP provider (such as a CBO) 210. The consumer 201, the DPP provider 210, the social support provider 220, and the payer 205 each interface with the integrator 203 at different to enroll the consumer 201 in an appropriate DPP and social solution, and thereafter perform program monitoring, completion, and payment. In some embodiments, the consumer 201 may be a candidate, a participant, a patient, or a user, as previously described.

The integrator 203 interfaces 231 with database 230 to identify a pool of candidates in a defined location. For example, the database 230 can include the entries of any number of different diseases and conditions from the BCBS database, which have been tagged by location. The integrator 203 can create a plurality of geospatial layers for a heat map, which includes the data and the locations associated with the 200 different diseases and conditions. In some examples, the heat map can include geospatial layers created by analyzing and aggregating a large database of claims data.

The database 230 can integrate DPP provider data, including the nature and location of local community based resources, with geographical data on disease metrics and social determinants to generate the heat map. Using the heat map, the integrator 203 can design an intervention policy for a defined location (for example, at the zip code or neighborhood level) to improve community health in the defined location. An analysis of the heat map produces data for the defined location, which can identify, for example, the highest health risks factors by future health costs, the highest risk factors by numbers of people, the effect of social determinants on the population, and/or the percentage of the population, which has access to either medical health insurance or a government program that pays for medical costs.

Using the results of the analysis, the integrator 203 knows the health risk factors to focus on in the defined location and the probability of reimbursement for providing a DPP to a candidate. In addition, these results can provide the integrator 203 an understanding of any social determinants, in the defined location, that may negatively affect a likelihood of a candidate successfully completing the DPP. With this understanding, the integrator 203 can design social programs and/or services to overcome one or more of the social determinants, which can be offered to a candidate enrolling in a DPP. In addition, these results can be used by the integrator 203 to plan on what programs, DPPs, social services, and/or CBOs should be added into the defined location to meet demand.

The integrator 203 can bundle the costs of the social programs and/or services with the costs of the DPP and send it to a payer 205 for reimbursement for all of the costs. Using this model, the integrator 203 can assure the payer 205 that the candidate is successfully completing the DPP, which will lower future medical costs incurred by payer 205 for the candidate's treatment of the disease or condition. With negotiated rates charged by the DPP providers and the social support providers to the integrator 203, the payer 205 pays rate for the bundled services, which is lower than directly paying a DPP provider for services for the candidate.

The data base 230 can include consumer information, which the integrator 203 can use to contact and enroll one or more potential candidates that were identified in the analysis of the heat map into an appropriate DPP designed for the prevention of a certain treatable condition.

Based on data from the analysis of the heat map, the integrator 203 can identify a pool of potential candidates in the defined location. The integrator 203 contacts 208 the consumer 201, who is one of the potential candidates in the defined location. The contact 208 by the integrator 203 initiates a sequence of events, which are collectively referred to herein as a transaction.

The consumer 201 responds to the integrator 203. The consumer 201 inputs data 202 responsive to a health risk assessment and a personal preference survey. These data 202 are analyzed by the integrator's computer system, for example by using algorithms to determine if the consumer 201 is eligible for a DPP. Data 202 may be in the form of Yes or No answers and can be computed by employing a finite state machine or a modification thereof.

If the consumer 201 is eligible for the DPP, then the integrator's computer system determines the best-fit DPP provider 210 using analytics to compare the personal preferences of the consumer 201 to ideal personal preferences for each of a plurality of DPP providers stored in the database 230. In addition, the consumer 201 can enter a location that the consumer 201 will be before traveling to the DPP provider 210, for example, home, work, school, the gym, or the office. The location of the consumer 201 is compared to a location of each of the plurality of DPP providers and then analyzed. Based on this analysis of personal preferences and locations, a best-fit DPP provider 210 is identified. In some analysis, only the personal preferences are used to determine a best-fit DPP 210.

In this context, the consumer personal profile data may include, for example, patient contact information (including zip code), demographics, socio-economic factors, psychographics, health information, health care utilization, claims data, electronic medical record data, prescription history, and purchasing data.

Using the results from the analysis of the heat map, the integrator 203 can identify the social determinants that affect the defined location. The consumer 201 inputs data 237 responsive to a social asset survey, which includes, among other factors, the social determinants identified in the heat map.

The data 237 are analyzed by the integrator's computer system, for example by using algorithms to determine if the consumer 201 has one or more social needs for success, which would increase a likelihood the consumer 201 will successfully complete the DPP. This data 237 may be in the form of Yes or No answers and can be computed by employing a finite state machine or a modification thereof.

If the consumer 201 is eligible for one or more social needs, then the integrator's computer system determines the best-fit social support provider 210 using analytics and interfacing 235 with a social support provider database 234. Based on this analysis of personal preferences and social needs, a best-fit social support provider 220 is identified.

The integrator 203 enrolls the consumer 201 in the best-fit DPP with the best-fit DPP provider 210. Notice of enrollment 204 is sent to the consumer 201. A second notice of enrollment 209 is sent to the best-fit DPP provider 210.

The integrator 203 enrolls the consumer 201 in the best-fit social solution with the best-fit social support provider 220. Notice of enrollment 236 is sent to the consumer 201. A second notice of enrollment 219 is sent to the best-fit social support provider 220.

Upon enrollment in the DPP and the social solution, the integrator 203 bundles the claim for the DPP and the claim for the social solution. The integrator 203 then sends a bundled claim 206 to payer 205. The payer 205 sends an approval or payment 207 to integrator 203.

The consumer 201 participates 213 in the DPP and the DPP provider 210 delivers the DPP content 212 to the consumer 201. The DPP provider 210 reports 214 progress and other data to the integrator 203. The social support provider 220 provides social solution 222, which fulfills a need 223 of the consumer 201. By fulfilling the need 223, the DPP provider 210 will typically report an improvement in attendance and participation in the DPP by the consumer 210. With the improvement in participation, the consumer 201 can meet DPP milestones in a timely manner.

The DPP provider 210 reports 214 progress and other data to the integrator 203. The social support provider 220 reports 224 impact of social solution and other data to integrator 203. Once the integrator 203 identifies that the consumer 201 has satisfied a milestone or completed the DPP, the integrator 203 sends a payment 215 to the DPP provider 210 and sent payment 225 to the social support provider 220, which ends the transaction or advances the transaction to the next milestone.

Some embodiments include an option of the integrator 203 interfacing with a third-party 238. The integrator 203 may receive a request from a third-party 238 to monitor the progress of the consumer 201. The integrator 203 can provide a report (for example, on the progress in a program or at completion of a program) for the third party 238. For example, a third party 238 can be, but is not limited to, a healthcare provider, a court, a family member, a life coach, a probation officer, a twelve step sponsor, a doctor, an employer, or a government agency.

In an exemplary embodiment, a consumer 201 logs onto integrator's website; the integrator 203 matches the consumer 201 to programs in which the consumer 201 is likely to be successful; the integrator 203 verifies consumer's eligibility for the programs, and verifies that the payer 205 (for example, consumer's heath plan or a government program) covers the programs for which the consumer 201 is eligible; integrator 203 enrolls consumer 201 with program provider 210; the integrator 203 monitors program progress, and can change the program provider 210, if appropriate.

Further in this embodiment, the integrator 203 queries whether consumer 201 is challenged by success metrics (for example, but not limited to: transportation, food insecurity, depression); integrator 203 contacts social support provider 220 to address challenges; the integrator 203 bundles the costs of the social services with the cost of the covered medical benefit, and submits the bundled claim to the payer 205; the integrator 203 pays the program provider 210 and the social support provider 220; the integrator 203 charts the consumer 201 progress including costs for social services; the integrator 203 analyzes the charts to confirm that overall costs to the plan for the community are reduced, and that overall community health is improved.

Various embodiments provide a computer system for enrolling a consumer 201 into a disease prevention program and a social resource plan designed to increase a likelihood of success by the consumer 201 in the disease prevention program.

In an exemplary embodiment, the computer system can be configured to perform the steps of: receiving contact from the consumer 201; performing a health risk assessment of the consumer 201; processing data 202 from the health risk assessment against a set of minimum criteria for a plurality of disease prevention programs; identifying a disease prevention program based on a result from the processing data; surveying the consumer 201 for personal preferences for the disease prevent program; surveying the consumer 201 for social needs for success in the disease prevention program; creating a matrix of personal preferences for the consumer 201 from surveying the consumer 201; creating a list of social needs for the consumer from surveying the consumer 201; comparing the matrix of personal preferences for the consumer 201 against an ideal matrix of personal preferences for a plurality of providers, which are in a database accessible by the computer system; determining a best-fit provider 210 for the consumer 201 based on comparing the matrix; prioritizing the list of social needs for the consumer 201 to determine a highest impact social need for increasing a likelihood of success by the consumer 201 in the disease prevention program; comparing the matrix of personal preferences and the highest impact social need against an ideal matrix of personal preferences and social needs for a plurality of social support providers, which are in a database accessible by the computer system; determining a social resource plan for fulfilling the highest impact social need and a best-fit social support provider 220 for the consumer 201 based on comparing the matrix; enrolling 204 the consumer 201 into the disease prevention plan with the best-fit disease prevention provider 210; and enrolling 236 the consumer 201 into the social resource plan with the best-fit social support provider 220.

The computer system can be configured to perform the steps of: bundling costs of the disease prevention program and the social resource plan; and invoicing 206 a payer 205 plan for a claim for the bundled costs. The computer system can be configured to perform the step of: receiving 207 a first payment for the claim from the payer 205.

The computer system can be configured to perform the steps of: tracking progress 214 of the consumer 201 in the disease prevention program and tracking progress 224 of the consumer 201 in the social resource plan; recognizing a milestone completed by the consumer 201 in the disease prevention program; sending a portion of the first payment 215 to the disease prevention program provider 210 upon recognizing the completed milestone; and sending a portion of the first payment 225 to the social support provider 220 upon recognizing the completed milestone.

The computer system can be configured to perform the step of: retaining a portion by the integrator 203 of the first payment for the claim.

Some embodiments provide a computer implemented method for using aggregate community health statistics to drive patient enrollment in disease prevention programs.

The method can include the steps of: analyzing a database comprising a plurality of community health statistics; generating a plurality of geospatial data layers of disease incidence from the plurality of community health statistics; creating a heat map comprising the plurality of geospatial data layers; segmenting the heat map into smaller defined areas; determining which of the defined areas have a high incidence of a preventable disease; analyzing a patient database comprising a plurality of patient names, each of the patient names tagged with a patient's address and the patient's contact information; identifying a group of patients in the defined areas with the high incidence of the preventable disease; contacting each of the identified patients in the group of patients; and enrolling at least a portion of the identified patients into a disease prevention program for the preventable disease.

The method can include the steps of: surveying each of the identified patients for personal preferences for the disease prevent program; and determining a best-fit disease prevention program provider for each of the identified patients based on comparing the personal preferences. The best fit disease prevention program provider is located in the defined area.

The method can include the steps of: submitting a claim for the disease prevention program for each of the identified patients to a group of payers; and receiving a payment for at least one claim from the group of payers.

The method can include the step of: sending a portion of the payment to a disease prevention program provider. The payment can include a premium paid to a system facilitator.

The method can include the steps of: opening an account for each of the identified patients enrolled in the disease prevention program; including a transaction fee for the account in the claim; and sending the transaction fee included in the payment to a system facilitator.

The method can include the steps of: monitoring progress in the prevention program for one of the identified patients; reporting achievement of a program milestone to the payer; and receiving payment from the payer. The payer can be a health insurance provider for the one identified patient.

The method can include the steps of: surveying each of the identified patients for social needs for success in the disease prevention program; determining a highest impact social need for increasing a likelihood of success in the disease prevention program for each of the identified patients; developing a best-fit social solution for the highest impact social need for each of the identified patients with a group of social support providers; determining a best-fit social support provider for each of the identified patients from the group of social support providers; and enrolling each of the identified patients in the best-fit social solution with the best-fit social support provider.

The method can include the steps of: bundling a claim for the disease prevention program and a claim for the best-fit social solution for each of the identified patients; sending a bundled claim for each of the identified patients to a group of payers; and receiving a payment for one of the bundled claims from the group of payers.

The method can include the steps of: sending a first portion of the payment to a disease prevention program provider; and sending a second portion of the payment to a social support provider. The method can include the step of: sending a third portion of the payment to a system facilitator.

The method can include the steps of: determining which of the defined areas have a high incidence of a second preventable disease; analyzing the patient database; identifying a second group of patients in the defined areas with the high incidence of the second preventable disease; contacting each of the identified patients in the second group of patients; and enrolling at least a portion of the identified patients in the second group of patients into a disease prevention program for the second preventable disease.

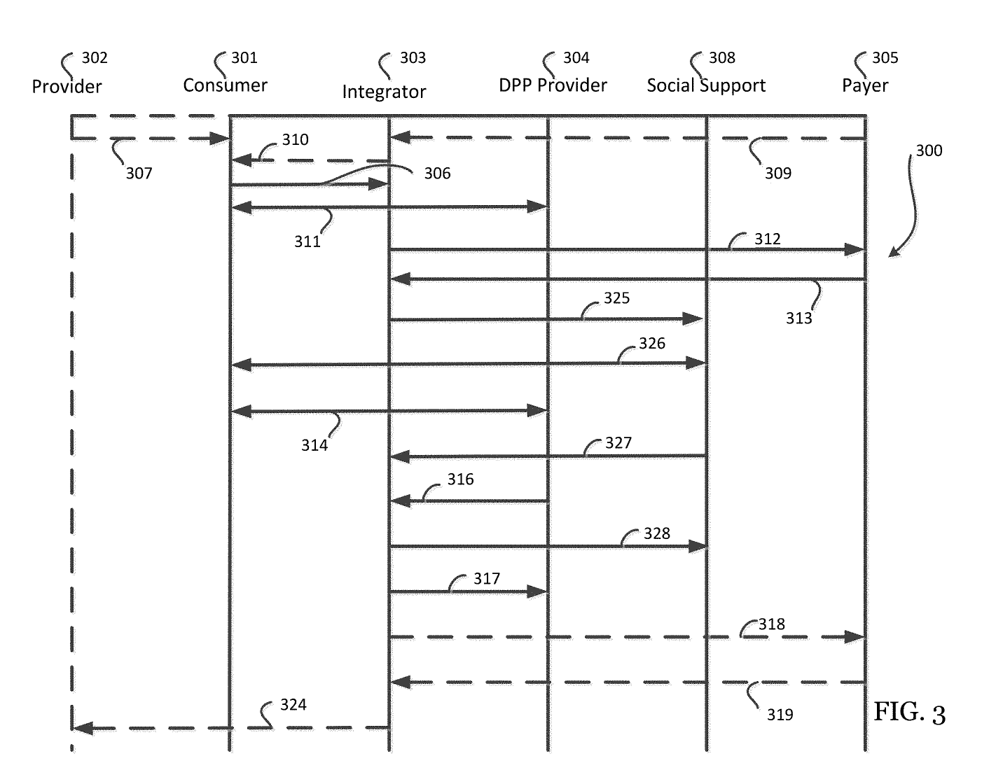

Turning to FIG. 3, a process flow diagram 300 illustrates an exemplary use case involving a consumer 301, an integrator 303, a DPP provider 304, a payer 305, a social support 308, and optionally a healthcare provider 302. All of these parties have been described in detail in various portions of this application.

Some embodiments include an option of the payer 305 (for example: a health care plan, an employer benefit plan, Medicare, and the like) referring a consumer 301 to the integrator 303 (Step 309), whereupon the integrator 303 contacts the consumer 301 and invites the consumer 301 to log into the integrator's system and find a DPP that is a best-fit for the consumer 301 (Step 310).

Some embodiments include an option of the healthcare provider 302 providing the consumer 301 with a prescription or other instructions to attend a DPP, which may include instructions for contacting the integrator 303 (Step 307).

In some embodiments the integrator 302 creates a heat map comprising of a plurality of geospatial layers of disease incidence. The integrator 302 can determine which of the defined areas have a high incidence of a preventable disease, and then analyze a candidate database comprising a plurality of names, each tagged with an address and contact information. The integrator 302 can identify a group of candidates in the defined areas with the high incidence of the preventable disease. The integrator 302 can contact each of the group of candidates, such as, consumer 301 (Step 310).

The consumer 301 contacts the integrator 302 through a portal and provides various data points, such as, for example, the DPP desired, personal information such as, name, address, zip code, associated payer 305 information, which is used to set up an account in the integrator's system (Step 306). The various data points can include the referral from Step 309 or the prescription from Step 307. The integrator 303 guides the consumer 301 through a survey to create a health risk assessment and a matrix of personal preferences, which can include preferred modes of content delivery, location, time/days for sessions, group dynamics, virtual options, and the consumer's level of motivation to complete the DPP. The health risk assessment can be designed to provide Yes or No answers to whether or not the consumer 301 meets the criteria to be entered into the desired DPP.

The integrator 303 guides the consumer 301 through a social asset survey. The social asset survey is analyzed determine if the consumer 301 has one or more social needs for success, which would increase a likelihood the consumer 301 will successfully complete the desired DPP.

The matrix of personal preferences generates a personality profile, preferably including a location. If the consumer is eligible for one or more DPPs, the personality profile can be mapped against a plurality of ideal personality profiles associated with the DPP providers 304.

Using the results of the personality profile analysis, the consumer's location (e.g., home or work address), and the social determinants, the integrator 303 determines which DPP provider 304 is the best-fit DPP provider 304 for the consumer 301. The integrator 303 enrolls the consumer 301 in the desired DPP with the best-fit DPP provider 304. The integrator 303 sends notice of the enrollment (which can include a DPP class schedule and any other information about the DPP, such as, dress code or dietary restrictions, or required monitoring systems) to both the consumer 301 and the DPP provider 304 (Step 311). However, if the consumer 301 is already affiliated with a particular health plan from the payer 305, the integrator 303 may permit the payer or the consumer to designate a preferred DPP provider 304, as the best-fit DPP provider 304 for one or more of the DPPs for which the consumer is eligible.

If the consumer 301 is eligible for one or more social needs, the integrator 303 reaches out (e.g., electronically over a network) to a social support provider 308 to address the identified needs (Step 325). The information and schedule of the social support can be included in the notice of enrollment of Step 311.

Upon the enrollment of the consumer 301, the integrator 303 prepares and sends a claim to the payer 305 (Step 312). The claim can include the bundling of costs of the DPP and the social support. The integrator 303 receives an approval of the bundled claim from the payer 305, which may include a partial payment claim (Step 313).

The social support provider 308 fulfills the consumer's identified social needs, which allows for participation in the DPP (Step 326). The consumer 301 participates in the DPP (Step 314). The DPP provider 304 provides the resources and delivers the content of the DPP to the consumer 301 (Step 315).

The social support provider 308 continually updates the integrator 303 (Step 327). As the consumer 301 progresses through the DPP, the DPP provider 304 updates the consumer's record and progress within a shared database maintained by the integrator 303 (Step 316).

In some embodiments, the integrator 303 may provide an interactive software tool for use by the DPP provider 304 to facilitate the integration process, for example, by allowing the DPP provider 304 to enter consumer data (e.g., attendance, body weight, and the like) directly into consumer's records maintained by, on behalf of, or at the direction of the integrator 303. In an embodiment, such an interactive software tool may include the Solera™ technology platform program available from Solera™ Health, Inc. located in Phoenix, Ariz.

Upon completion of the DPP or, alternatively, at various predetermined milestones, the integrator 303 makes a partial or full payment to the DPP provider 304 (Step 317). In addition, integrator 303 makes a partial or full payment to the social support provider 308 (Step 328).

In some embodiments, if multiple milestones are required for program completion, the system 300 can be setup to prepare and send one or more interim or supplemental claims to the payer 305 upon completion of each milestone (Step 316). The integrator 303 receives an approval of the interim or supplemental claim, which may include a partial payment. The DPP provider 304 continues to update the consumer's record and progress within the shared database maintained by the integrator 303. The social support provider 308 continues to update the integrator 303 (Step 327). Upon completion of the DPP or, alternatively, at the next predetermined milestone, the integrator 303 makes an additional partial or final payment to the DPP provider 304 (Step 317) and to the social support provider 308 (Step 328). These Steps can be repeated multiple times, as determined by the number of milestones that are in a particular DPP.

In some embodiments, the integrator 303 may send the consumer 301 a survey or otherwise solicit feedback at certain times during the DPP. The consumer 301 completes the survey and the survey results are stored by the integrator 303. The survey can be directed to the quality and efficiency of the DPP and/or the DPP provider. Using machine learning capabilities of the integrator system, the results of a group of surveys can be analyzed to modify the ideal personality profile and/or other metrics for the DPP provider 304. In addition, the results of a group of surveys can be used to rank the DPP provider 304 among various DPP providers in a network.

In another embodiment, the consumer 301 can track data and milestones by accessing a dashboard provided by the integrator 303. The integrator 303 continually updates the data and populates the fields in the dashboard for viewing by the consumer 301. In some embodiments, the integrator 303 can send one or more reports regarding the consumer's progress to the medical healthcare provider 302 (Step 324). The report can confirm successful completion of the DPP by the consumer 301 or, alternatively, can report the status if the DPP was not successfully completed. In this way the healthcare provider 302 can report aggregate quality metrics to Medicare/Medicaid agencies and the CDC, as well as chart and report the consumer's performance to the DPP.

In some embodiments, the integrator 303 reserves a portion of the payment from the payer 305, as compensation to the integrator for facilitating and managing the process. Alternatively, the payer 305 may pay a premium over the standard rate for the bundled claim in order to compensate the integrator 303 facilitating and managing the process. Typically, the premium paid by the payer 305 is less than the cost that the payer 305 would otherwise incur to facilitate and manage the process if the integrator 303 were not used, thus resulting in a net cost saving for the payer 305 in any event. In some embodiments, a set-up fee or a records fee may be included in the claim sent to the payer 305. This fee reimburses the integrator 303 for the costs of enrolling the consumer 301 in the DPP provided by the best-fit DPP provider and initiating an account for the consumer 301.

Various embodiments provide methods performed by a computer system 300 for enrolling a consumer 301 into a disease prevention program and a social resource plan configured to increase a likelihood of success in the program.

In an exemplary embodiment, a method can include the steps of: receiving contact from the consumer 301 (Step 306); performing a health risk assessment of the consumer 301; processing data from the health risk assessment against a set minimum criteria for a plurality of disease prevention programs; identifying a disease prevention program based on a result from the processing data; surveying the consumer 301 for personal preferences for the disease prevent program; surveying the consumer 301 for social needs for success in the disease prevention program; creating a matrix of personal preferences for the consumer 301 from surveying the consumer 301; creating a list of social needs for the consumer 301 from surveying the consumer 301; comparing the matrix of personal preferences for the consumer 301 against an ideal matrix of personal preferences for a plurality of providers, which are in a database accessible by the computer system; determining a best-fit provider 304 for the consumer 301 based on comparing the matrix; prioritizing the list of social needs for the consumer 301 to determine a highest impact social need for increasing a likelihood of success in the disease prevention program; comparing the matrix of personal preferences and the highest impact social need against an ideal matrix of personal preferences and social needs for a plurality of social support providers, which are in a database accessible by the computer system; determining a social resource plan for fulfilling the highest impact social need and a best-fit social support provider 308 for the consumer 301 based on comparing the matrix; enrolling the consumer 301 into the disease prevention plan with the best-fit disease prevention provider 304 (Step 311); and enrolling the consumer 301 into the social resource plan with the best-fit social support provider 308 (Step 326).

The method can include the step of: bundling costs of the disease prevention program and the social resource plan. The method can include the step of: invoicing the payer 305 for a claim for the bundled costs (Step 312).

The method may include receiving a first payment for the claim from the payer 305, which can be the consumer's health insurance plan (Step 313).

In some configurations, the method includes the steps of: tracking progress of the consumer 301 in the disease prevention program and the social resource plan; recognizing a milestone completed by the consumer in the disease prevention program (Step 316); sending a portion of the first payment to the disease prevention program provider 304 upon recognizing the completed milestone (Step 317); and sending a portion of the first payment to the social support provider 308 upon recognizing the completed milestone (Step 328).

The method may also include the step of providing the consumer 301 a notice of enrollment in the disease prevention program with the best-fit program provider 304 (Step 311). The method can include the step of: providing the best-fit program provider 304 a notice of enrollment of the consumer 301 in the disease prevention program (Step 311).

The method can include the step of: providing the consumer 301 a notice of enrollment in the social resource plan with the best-fit social support provider 308 (Step 311). The method can include the step of: providing the best-fit social support provider 306 a notice of enrollment of the consumer 301 in the social resource plan (Step 325).

The method can include the step of: retaining a portion by the integrator 303 of the first payment for the claim from the payer 305. The first payment for the claim includes a premium paid to the integrator 303. The first payment for the claim includes a records fee for opening an account for the consumer 301, which retained by the integrator 303.

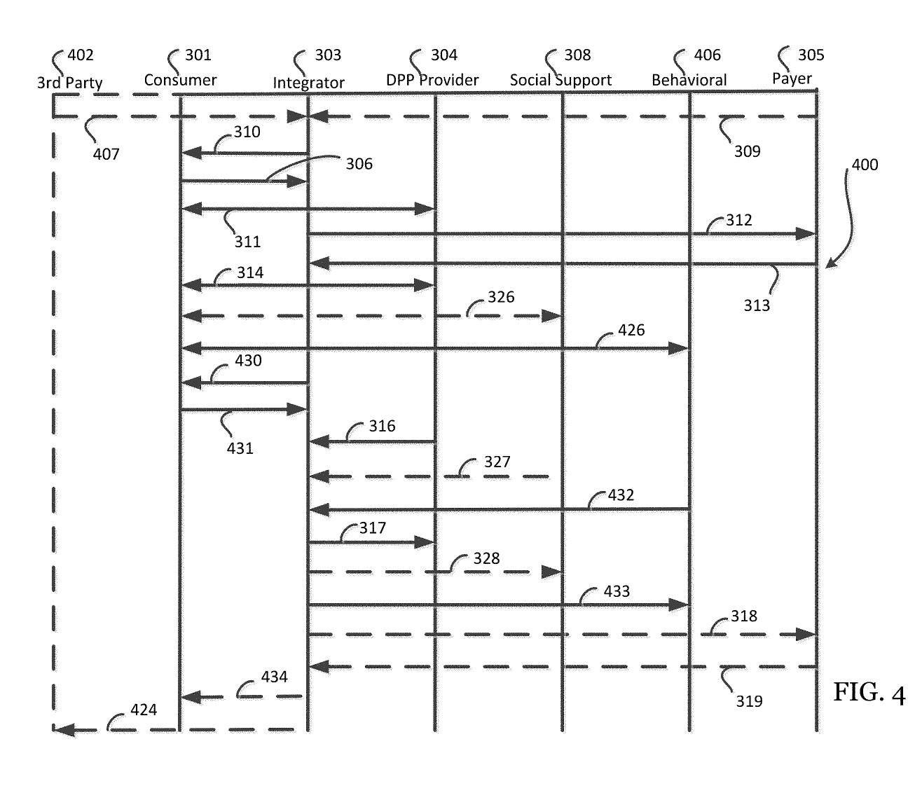

FIG. 4 is another process flow diagram 400 illustrating an exemplary use case involving a consumer 301, an integrator 303, a DPP provider 304, a social support provider 308, a behavioral services provider 406, a payer 305, and optionally a third party 402. All of these parties have been described in detail in various portions of this application. For brevity, the steps described for FIG. 3 will not be repeated for the same steps in description of FIG 4.

Some embodiments include an option of a third party 402 providing the integrator 303 with request to enroll the consumer 301 in one or more programs. The request can include contact information, a prescription for at least one DPP, a court order for a behavioral program, a need for a social solution, or other instructions directed to the consumer' needs, as determined by the third party (Step 407).

As described herein, the integrator 303 guides the consumer 301 through a survey to create a health risk assessment and a matrix of personal preferences, which can include preferred modes of content delivery, location, time/days for sessions, group dynamics, virtual options, and the consumer's level of motivation to complete the DPP. The health risk assessment can be designed to provide Yes or No answers to whether or not the consumer 301 meets the criteria to be entered into the desired DPP.

The integrator 303 guides the consumer 301 through a behavioral health assessment. The behavioral health assessment is analyzed determine if the consumer 301 should participate in a behavioral health plan, which would increase a likelihood the consumer 301 will successfully complete the desired DPP.

The matrix of personal preferences generates a personality profile. If the consumer 301 is eligible for one or more DPPs, the personality profile can be mapped against a plurality of ideal personality profiles associated with the DPP providers 304. Using the results of the personality profile analysis, the consumer's location, the integrator 303 determines which DPP provider 304 is the best-fit DPP provider 304 and a best-fit behavioral support provider 406 for the consumer 301.

The integrator 303 enrolls the consumer 301 in the desired DPP with the best-fit DPP provider 304. The integrator 303 enrolls the consumer 301 in a behavioral health plan with the best-fit behavioral support provider 406. The integrator 303 sends notices of the enrollment to the consumer 301, the DPP provider 304, and the behavioral support provider 406 (Step 311).

Upon the enrollment of the consumer 301, the integrator 303 prepares and sends a claim to the payer 305 (Step 312). The claim can include the bundling of costs of the DPP and the behavioral health plan. The integrator 303 receives an approval of the bundled claim from the payer 305, which may include a partial payment claim (Step 313).

If a social need is identified, a social support provider 308 fulfills the consumer's identified social needs, which allows for participation in the DPP (Step 326). The integrator 303 prepares and sends a claim, which bundles costs of the DPP, the behavioral health plan, and the social support, to the payer 305 (Step 312). The integrator 303 receives an approval of the bundled claim from the payer 305, which may include a partial payment claim (Step 313).

The behavioral support provider 406 continually updates the integrator 303 on the progress of the behavioral health plan (Step 427). As the consumer 301 progresses through the DPP, the DPP provider 304 updates the consumer's record and progress within a shared database maintained by the integrator 303 (Step 316).

Upon completion of the DPP or, alternatively, at various predetermined milestones, the integrator 303 makes a partial or full payment to the DPP provider 304 (Step 317). In addition, integrator 303 makes a partial or full payment to the behavioral support provider 406 (Step 428). If the social need has been addressed, integrator 303 makes a partial or full payment to the social support provider 308 (Step 328).

In some embodiments, if multiple milestones are required for program completion, the system 300 can be setup to prepare and send one or more interim or supplemental claims to the payer 305 upon completion of each milestone (Step 316). The integrator 303 receives an approval of the interim or supplemental claim, which may include a partial payment. The DPP provider 304 continues to update the consumer's record and progress within the shared database maintained by the integrator 303. The behavioral support provider 406 continues to update the integrator 303 (Step 427). Upon completion of the DPP or, alternatively, at the next predetermined milestone, the integrator 303 makes an additional partial or final payment to the DPP provider 304 (Step 317) and to the behavioral support provider 406 (Step 328). These Steps can be repeated multiple times, as determined by the number of milestones that are in a particular DPP.

In some embodiments, the integrator 303 may send the consumer 301 a survey or otherwise solicit feedback at certain times during the DPP (Step 430). The consumer 301 completes the survey and the survey results are stored by the integrator 303 (Step 431). The survey can be directed to the quality and efficiency of the DPP and/or the DPP provider. Using machine learning capabilities of the integrator system, the results of a group of surveys can be analyzed to modify the ideal personality profile and/or other metrics for the DPP provider 304. In addition, the results of a group of surveys can be used to rank the DPP provider 304 among various DPP providers in a network.

In another embodiment, the consumer 301 can track data and milestones by accessing a dashboard provided by the integrator 303 (Step 434). The integrator 303 continually updates the data and populates the fields in the dashboard for viewing by the consumer 301. In some embodiments, the integrator 303 can send one or more reports regarding the consumer's progress to the third party 402 (Step 424).

Various embodiments provide methods performed by a computer system 400 for enrolling a consumer 301 into a disease prevention program and a behavioral health plan configured to increase a likelihood of success in the program.

In an exemplary embodiment, the method includes the steps of: receiving contact from the consumer 301 (Step 306); performing a health risk assessment of the consumer 301; processing data from the health risk assessment against a set minimum criteria for a plurality of disease prevention programs; identifying a disease prevention program based on a result from the processing data; surveying the consumer 301 for personal preferences for the disease prevent program; performing a behavioral health assessment; creating a matrix of personal preferences for the consumer 301; comparing the matrix of personal preferences for the consumer 301 against an ideal matrix of personal preferences for a plurality of providers, which are in a database accessible by the computer system; determining a best-fit provider 304 and a best-fit behavioral health provider 406 for the consumer 301 based on comparing the matrix; enrolling the consumer 301 into the disease prevention plan with the best-fit disease prevention provider 304 (Step 311); and enrolling the consumer 301 into the behavioral health plan with the best-fit behavioral support provider 406 (Step 426).

The method can include the step of: bundling costs of the disease prevention program and the behavioral health plan. The method can include the step of: invoicing the payer 305 for a claim for the bundled costs (Step 312). The method can include the step of: receiving a first payment for the claim from the payer 305, which can be the consumer's health insurance plan (Step 313).

In some configurations, the method can include the steps of: tracking progress of the consumer 301 in the disease prevention program and the behavioral health plan; recognizing a milestone completed by the consumer in the disease prevention program (Step 316); sending a portion of the first payment to the disease prevention program provider 304 upon recognizing the completed milestone (Step 317); and sending a portion of the first payment to the behavioral support provider 406 upon recognizing the completed milestone (Step 428).

The method can include the steps of: surveying the consumer 301 for social needs for success in the disease prevention program; creating a matrix of personal preferences for the consumer 301 from surveying the consumer 301; creating a list of social needs for the consumer 301 from surveying the consumer 301; prioritizing the list of social needs for the consumer 301 to determine a highest impact social need for increasing a likelihood of success in the disease prevention program; comparing the matrix of personal preferences and the highest impact social need against an ideal matrix of personal preferences and social needs for a plurality of social support providers, which are in a database accessible by the computer system; determining a social resource plan for fulfilling the highest impact social need and a best-fit social support provider 308 for the consumer 301 based on comparing the matrix; and enrolling the consumer 301 into the social resource plan with the best-fit social support provider 308 (Step 326).

The method can include the step of: bundling costs of the disease prevention program, the behavioral health plan, and the social resource plan. The method can include the step of: invoicing the payer 305 for a claim for the bundled costs (Step 312).

The method can include the steps of: tracking progress of the consumer 301 in the disease prevention program and the social resource plan; recognizing a milestone completed by the consumer in the disease prevention program (Step 316); sending a portion of the first payment to the disease prevention program provider 304 upon recognizing the completed milestone (Step 317); sending a portion of the first payment to the social support provider 308 upon recognizing the completed milestone (Step 328) and sending a portion of the first payment to the behavioral support provider 406 upon recognizing the completed milestone (Step 428).

Some embodiments provide computer code stored in a non-transient medium for performing, when executed by a computer processor, the steps of: receiving contact from the consumer 301 (Step 306); performing a health risk assessment of the consumer 301; processing data from the health risk assessment against a set minimum criteria for a plurality of disease prevention programs; identifying a disease prevention program based on a result from the processing data; surveying the consumer 301 for personal preferences for the disease prevent program; performing a behavioral health assessment; creating a matrix of personal preferences for the consumer 301; comparing the matrix of personal preferences for the consumer 301 against an ideal matrix of personal preferences for a plurality of providers, which are in a database accessible by the computer system; determining a best-fit provider 304 and a best-fit behavioral health provider 406 for the consumer 301 based on comparing the matrix; enrolling the consumer 301 into the disease prevention plan with the best-fit disease prevention provider 304 (Step 311); and enrolling the consumer 301 into the behavioral health plan with the best-fit behavioral support provider 406 (Step 426).

The computer code, when executed by a computer processor, can further comprise the steps of: bundling costs of the disease prevention program and the behavioral health plan; and invoicing the payer 305 for a claim for the bundled costs (Step 312).

The computer code, when executed by a computer processor, can further comprise tracking progress of the consumer 301 in the disease prevention program and the behavioral health plan; recognizing a milestone completed by the consumer in the disease prevention program (Step 316); sending a portion of the first payment to the disease prevention program provider 304 upon recognizing the completed milestone (Step 317); and sending a portion of the first payment to the behavioral support provider 406 upon recognizing the completed milestone (Step 428).

In accordance with various embodiments, an integrator system can be configured to programmatically determine an ideal personality profile based on the outcomes of a population subset in a defined area for each DPP provider. Each DPP provider can have one or more vehicles for delivering a DPP. For example, a DPP provider can offer a qualifying DPP in a group session or can offer another qualifying DPP using a virtual interface.

In some cases, a consumer may be in need of more than one DPP. For example, a consumer may be in need DPPs for one or more of congestive heart failure (“CHF”), coronary artery disease (“CAD”), type-2 diabetes, depression, chronic obstructive pulmonary disease (“COPD”), hypertension, and hyperlipidemia. The integrator system can include a patient health risk stratification system configured to recognize more than one chronic disease and determine the highest priority chronic diseases for a candidate consumer. In one embodiment, a consumer's health risk assessment is driven by machine learning to analyze previous answers and determine if additional question strings need to be added to the assessment, which evaluate the consumer for other disease conditions.

The integrator system can be configured use a list of the highest priority chronic diseases to identify a corresponding DPP for each chronic disease on the list. The integrator system can be configured to administer multiple DPPs for a consumer. Depending of the group of multiple DPPs, the timing of the consumer taking each DPP may be simultaneously (all at once), sequentially, overlapping, or some together and others later in time. The integrator system can be configured to administer the scheduling and coordinating any timing structure for a consumer taking a group of multiple DPPs.

Various embodiments provide a Precision Prevention Network, which imports data related to a consumer's unique prevention profile to create a personalized dashboard for not only qualified DPPs, but also the type and delivery intervention method of the DPPs based on their unique needs and preferences. The Network can create a precision prevention plan for a consumer that predicts the best DPPs and DPP providers based on any or all of the following factors: demographics, medical information, co-morbidities, social determinants needs, program availability, patient motivation, learning environment, frequency of provider touch points with patient, language, and cultural competency. In some embodiments, a healthcare provider could transition a consumer to the Network, which would then manage the consumer between episodes of clinical care based on the consumer's precision prevention plan.

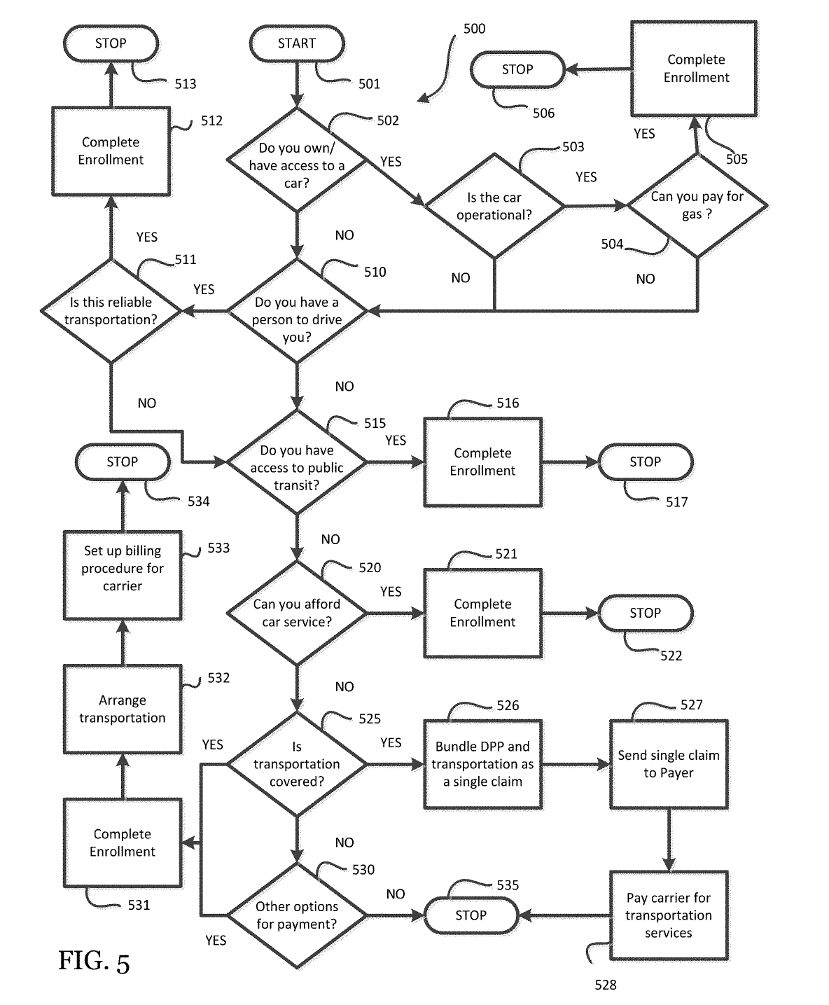

Moving on to FIG. 5, a flow chart illustrates exemplary steps of a process 500 for determining whether a consumer is in need of a social resource to increase a likelihood of success in a DPP. In this example, the social resource is transportation to and from the DPP. This example can be one part of the social asset survey used during the enrollment process for the DPP. If a need for this social resource is met, the consumer continues through the process and completes enrollment into the DPP and a social resource plan. The process 500 will be described below in the context of a second-person narrative, asking a series of questions in the way it might be presented to the consumer.

The process 500 starts 501 with a first question: do you own or have access to a car? (Step 502). If the answer is NO, then move to Step 510, which is described below. If the answer is YES, then is the car operational? (Step 503). If NO, move to Step 510. If YES, can you pay for gas? (Step 504). If NO, move to Step 510. If YES, complete enrollment (Step 505) and stop 506.

Now moving to Step 510, the system queries whether the patient has a person to drive him or her to and from the DPP. (Step 510). If NO, move to Step 515, which is described below. If YES, is the transportation reliable? (Step 511). If NO, move to Step 515. If YES, complete enrollment (Step 512) and stop 513.

Moving to Step 515, do you have access to public transportation? (Step 515). If YES, complete enrollment (Step 516) and stop 517. If NO, can you afford car service (for example, a taxi, Uber, or Lyft)? (Step 520). If YES, complete enrollment (Step 521) and stop 522. If NO, is transportation to and from DPP covered by consumer's payer? (Step 525) If YES, move to Step 531, which is described below. If YES, bundle DPP costs and transportation costs into a single claim (Step 526). Then send bundled claim to the payer (Step 527) and pay carrier/provider from claim payment after serves are rendered (Step 528) and stop 535.

If NO, are there other options for payment for transportation? (Step 530). If NO, stop 535. If YES, complete enrollment (Step 531). Arrange transportation for consumer to and from the DPP with a carrier/provider (Step 532). Then set up billing procedure with carrier/provider (Step 533) and stop 534.

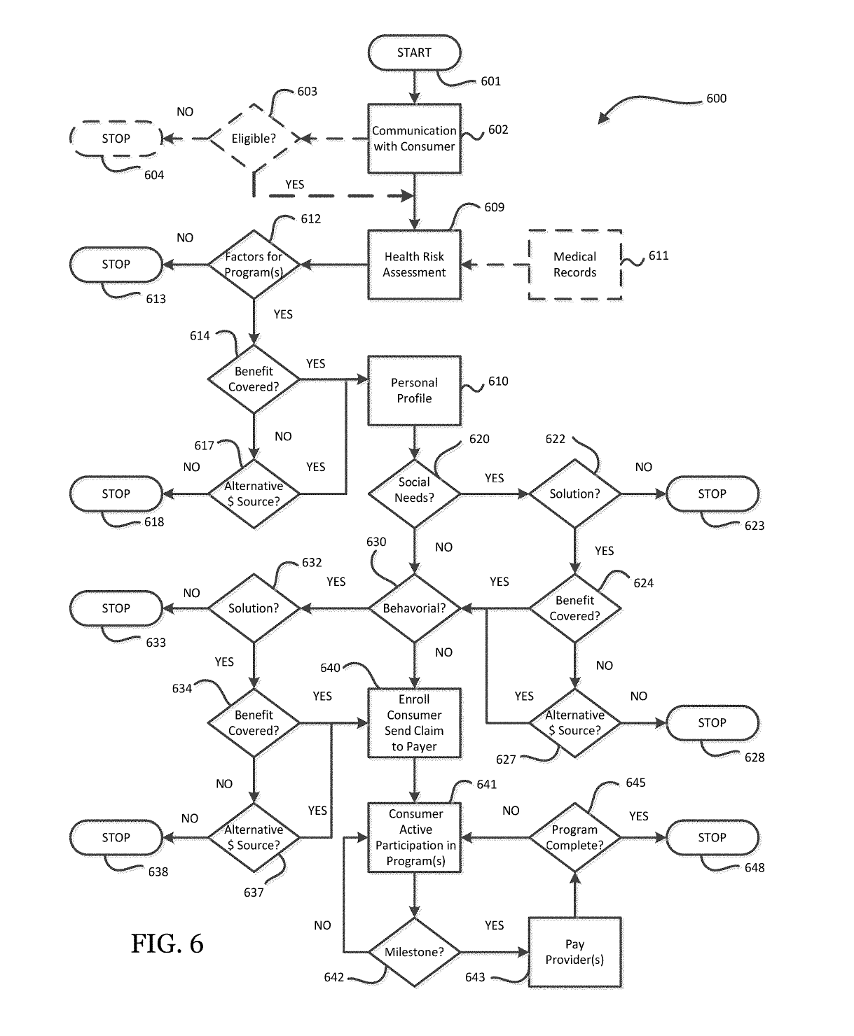

FIG. 6 is a flow chart illustrating exemplary steps for a process 600 for enrolling a consumer by an integrator. The process 600 starts 601 with communication with the consumer (Step 602). During this communication, integrator can determine if the consumer is eligible for a program (Step 603). If NO, then stop 604. If Yes, perform a health risk assessment on consumer (Step 609). The health risk assessment can optionally include accessing consumer's medical records (Step 611). From the health risk assessment, certain risk factors identified. Are the risk factors at levels high even for enrollment in one or more DPPs? (Step 612). If NO, then stop 613. If YES, does the consumer's benefit plan cover the one or more DPPs? (Step 614). If YES, move to Step 610. If NO, is there an alternative payment method? (Step 617). If NO, then stop 618. If YES, the consumer enters data for a personal profile (Step 610).

Moving to the next step from the personal profile (Step 610), any social needs that would increase success in the programs? (Step 620). If YES, is a reasonable solution to fulfill the social need available? (Step 622). If NO, then stop 623. If YES, does the consumer's benefit plan cover the social resource solution? (Step 624). If YES, move to Step 630. If NO, Is there an alternative payment method? (Step 627). If YES, move to Step 630. If NO, then stop 628.

If NO for the social needs (620), any behavioral health needs that would increase success in the programs? (Step 630). If YES, is a reasonable solution to the behavioral health need available? (Step 632). If NO, then stop 633. If YES, does the consumer's benefit plan cover the solution? (Step 634). If YES, move to Step 640. If NO, is there an alternative payment method? (Step 637). If YES, move to Step 640. If NO, then stop 638.

If NO for the behavioral health needs (630), enroll the consumer in one or DPPs and additionally in a social resource plan and/or a behavioral health plan, as appropriate, then send claim or bundled claim to payer (Step 640). The consumer actively participates in the programs (Step 641). As the consumer progresses through the program, is a milestone hit? (Step 642). If NO, go back to Step 641. If YES, pay provider(s), which can include one or more DPP providers, and may include a social support provider, and/or a behavioral health provider (Step 643). Is the program complete? (Step 645). If NO, return to Step 641. If YES, then stop 648.

In some embodiments, the aggregate data drives the build out of the CBO network. For example, an integrator would enter the neighborhood and then contact all of the CBOs, such as, but not limited to, churches, blood centers, Jewish community centers, Spanish community centers, YMCAs, Walmart health and wellness neighborhood hubs. The integrator can identify CBOs with specific programs calculated to address known need. As demand changes, the integrator has the control to turn on or off these specific programs in the neighborhood.

In some embodiments, the integrator sets up relationships between the integrator, payers, and providers. The integrator can register participants referred by payers and/or employers. The integrator sets up relationships with CBO DPP providers for payment from plan routed thru the integrator. The integrator can create dashboard/heat map from zip code data. Based on the heat map, the integrator can notify the participants of the CBO resources. The integrator can develop and/or build out the CBO network based on the dashboard. The integrator can bundle claims for non-clinical services with clinical services and receive payment for the bundled claims from the payer.

In some embodiments, the intersection of a participant database with the aggregate community health database involves: analyzing a large database of claims data: generating heat maps of which zip codes/neighborhoods have a high incidence of certain treatable conditions; making recommendations to a plan as to what programs, social services, and/or CBOs need to be built out on a zip code/neighborhood basis; using the participant database participant to contact and enroll eligible participants in the appropriate programs.

The aggregate community health database includes the number of people in a zip code who are in need of a disease prevention program. However, the aggregate community health database cannot identify who they are. The participant database has access to the medical records from providers, employers, and consumers. The participant database can identify individuals in the zip code who are in need of a disease prevention program. The participant database has the ability to contact these individuals and enroll them in the DPP and into a social resource plan, if needed.

Some embodiments provide a computer implemented method for using aggregate community health statistics and demographics to identify eligible participants for disease prevention programs.

The method can include the steps of: analyzing a database comprising a plurality of community health statistics; generating a plurality of health geospatial data layers of disease incidence from the plurality of community health statistics; analyzing a database comprising a plurality of demographic statistics; generating a plurality of demographic geospatial data layers of demographic factors from the plurality of demographic statistics; creating a heat map comprising the plurality of geospatial data layers and the demographic geospatial data layers; segmenting the heat map into smaller defined areas; analyzing a database of eligible participants, the database comprising a plurality of participant names, each of the participant names is tagged with a participant address and contact information; identifying a group of eligible participants in each of the defined areas; and contacting the group of eligible participants.

The method can include the steps of: determining which of the defined areas have a high incidence of a preventable disease; identifying the group of eligible participants in the defined areas with the high incidence of the preventable disease; and enrolling at least a portion of the group of eligible participants into a disease prevention program for the preventable disease.

The method can include the steps of: analyzing disease incidence for a plurality of preventable conditions in each of the defined areas identifying a highest impact preventable condition for each of the defined areas; identifying the group of eligible participants with risk for the highest impact preventable condition in each of the defined areas; and enrolling at least a portion of the group of eligible participants into a disease prevention program for the preventable condition.

The method can include the steps of: determining which of the defined areas have a high incidence of a preventable disease; determining a set of demographic factors related to the incidence of the preventable disease; identifying the group of eligible participants having the set of demographic factors; and enrolling at least a portion of the group of eligible participants into a disease prevention program for the preventable condition.

The method can include the steps of: determining which of the defined areas have a high incidence of a preventable disease; identifying the group of eligible participants having the participant address in the defined areas; and enrolling at least a portion of the group of eligible participants into a disease prevention program for the preventable condition.

The method can include the steps of: preforming a health risk assessment for all of the eligible participants; determining which of the defined areas have a high incidence of a preventable disease; identifying a set of risk factors related to the preventable disease; analyzing the health risk assessment for the set of risk factors for all of the eligible participants; identifying the group of eligible participants having the set of risk factors in the defined areas; and enrolling at least a portion of the group of eligible participants into a disease prevention program for the preventable condition.

The method can include the steps of: identifying a hot spot defined area; determining a set of demographic factors associated with the hot spot defined area; identifying the group of eligible participants the set of demographic factors; and enrolling at least a portion of the group of eligible participants into a disease prevention program.

The method can include the steps of: identifying a hot spot defined area; identifying the group of eligible participants having an address in the hot spot defined area; and enrolling at least a portion of the group of eligible participants into a disease prevention program.

The method can include the steps of: enrolling at least a portion of the group of eligible participants into a disease prevention program; submitting a claim for the disease prevention program for each participant in the portion of the group of eligible participants to a group of payers; receiving a payment for at least one claim from the group of payers; and sending a portion of the payment to a disease prevention program provider.

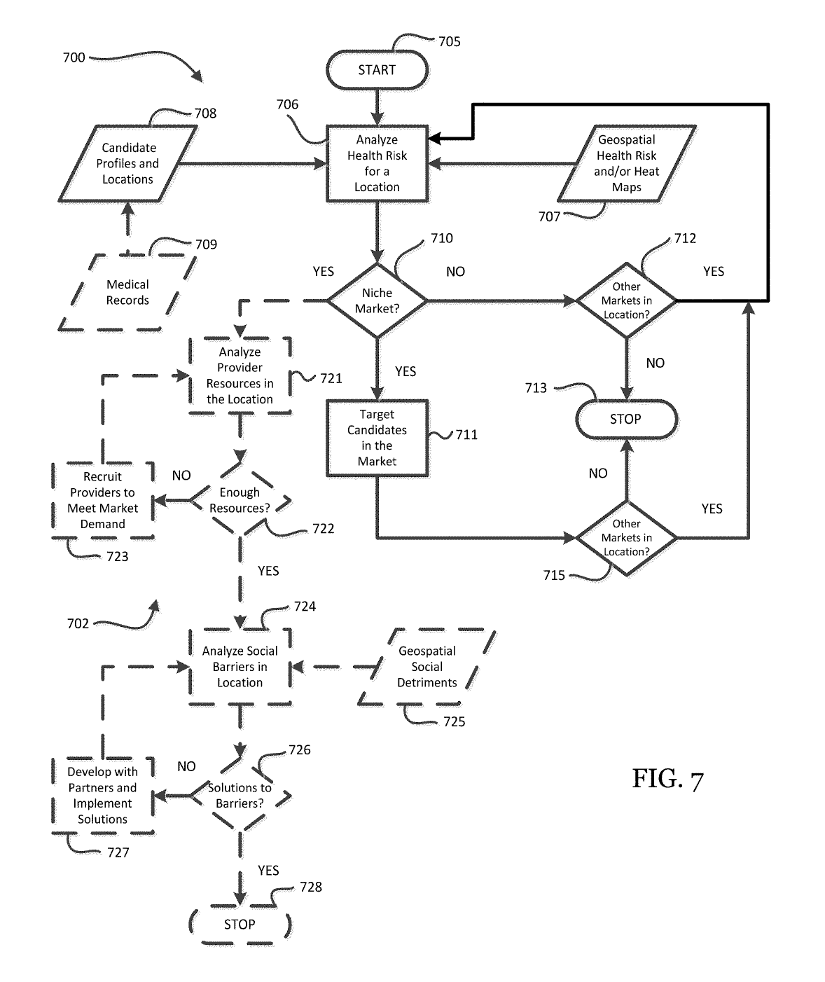

Turning to FIG. 7, a flow chart illustrates exemplary steps of a process 700 for analyzing data to determine a market in a location and targeting candidates in the market.

The process 700 starts 705 with analyzing heath risk for a location (Step 706). The location can be a defined area, as described herein. The location can be a zip code. A database comprising geospatial health risk data and/or heat maps for the location (Database 707) is used in Step 706. In addition, a database comprising candidate profiles and location (Database 708) is used in Step 706. A database comprising medical records (Database 709) can be added to Database 708.

The analyzing health risk of Step 706 can use one or more of the methods or techniques described herein, or any other appropriate method or technique now known or developed in the future. The analysis of Step 706 provides the data to determine whether or not a niche market exists in the location (Step 710).

A niche market can be for any one of the programs, as described herein. A niche market can be for a DPP or a particular combination of DDPs. A niche market can be for treating or preventing a disease with an abnormally high incidence rate in the location. A niche market can be for a DPP that the integrator does not provide in the location. In some configurations, a niche market can be identified as a hot stop on a heat map.

If NO, query for other niche markets in the location? (Step 712). If YES, go back to Step 706 and continue the analysis for other conditions or diseases. If NO, then stop 713.

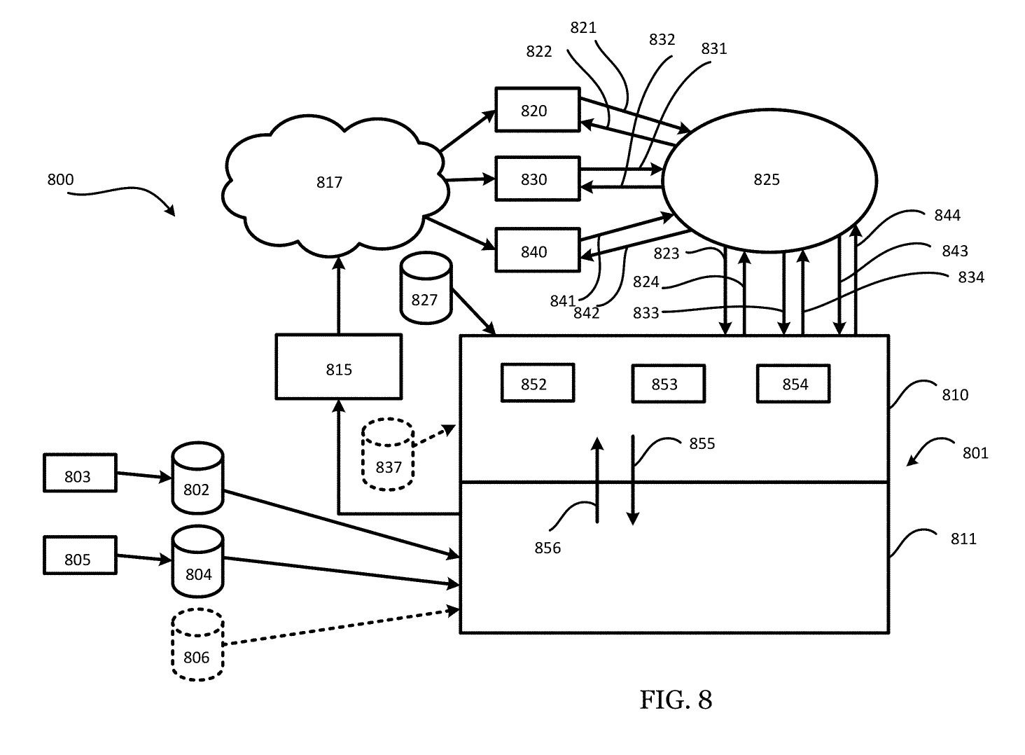

If YES, an integrator system targets candidates that make up the niche market (Step 711). The system can make contact with the candidates in any one or more of the methods or techniques described herein, or any other appropriate method or technique now known or developed in the future. In some configurations, the integrator system can be the system of FIG. 8, as described below.

Then query for other niche markets in the location? (Step 715). If YES, go back to Step 706 and continue the analysis for other conditions or diseases. If NO, then stop 713.

Optionally if YES, analyze the provider resources for the niche market in the location (Step 721). Do enough provider resources exist for the number of candidates that make up the niche market? (Step 722). If NO, recruit additional providers for the location to meet the market demand (Step 723), then go back to Step 721 and re-evaluate.

If YES, analyze potential social barriers and social determinants in the location, which may affect a likelihood of candidate being successful in the program (Step 724). If any social barriers are identified, are solutions available? (Step 726). If NO, develop solutions with social support providers and implement the solutions (Step 727), then go back to Step 724 and re-evaluate. If YES, then stop 728.

The process 700 can be computer automated. In some configurations, the location can be an individual candidate's location and the process 700 can generate a personal precision prevention plan for the individual candidate.

Some embodiments provide a computer implemented method for determining a need for disease prevention programs in a defined area.

The method can include the steps of: analyzing a database comprising a plurality of community health statistics; generating a plurality of geospatial data layers of incidence of disease from the plurality of community health statistics; creating a heat map comprising the plurality of geospatial data layers; segmenting the heat map into smaller defined areas; determining which of the defined areas have a high incidence of a disease; analyzing a consumer database comprising a plurality of consumer names, each of the consumer names tagged with a consumer's address, the consumer's health risk assessment, and the consumer's contact information; identifying consumers in the defined area that have an above average risk for the disease from the consumer's health risk assessment; identifying all disease prevention program providers in the defined area that are qualified to provide a prevention program for the disease; calculating a total consumer capacity of all of the prevention programs for the disease in the defined area; and comparing a total number of the consumers identified with an above average of risk for the disease to the total consumer capacity of all of the prevention programs for the disease in the defined area.

The method can include the steps of: receiving a result of the total number of consumers is greater than the total consumer capacity; and increasing an amount of the disease prevention program providers in the defined area that are qualified to provide the prevention program for the disease.

The method can include the steps of: contacting a portion of the consumers identified with an above average of risk for the disease; and enrolling at least a portion of the consumers contacted into the prevention program for the disease in the defined area. In some embodiments, the portion of consumers contacted is less than or equal to the total consumer capacity.

The method can include the steps of: submitting a claim for each of the consumers to a payer; and receiving payment for at least one claim from the payer.

The method can include the steps of: monitoring progress in the prevention program for one of the consumers; reporting achievement of a program milestone to the payer; and receiving payment from the payer.

The method can include the steps of: receiving a result of the total number of consumers is less than or equal to the total consumer capacity; contacting the consumers identified with an above average of risk for the disease; and enrolling at least a portion of the consumers in the prevention program for the disease in the defined area.

The method can include the steps of: submitting a claim for each of the consumers to a payer; and receiving payment for at least one claim from the payer.

The method can include the steps of: monitoring progress in the prevention program for one of the consumers; reporting achievement of a program milestone to the payer; and receiving payment from the payer.

The method can include the steps of: monitoring at least one consumer in the defined area; receiving a signal the consumer is at above average risk for the disease; enrolling the consumer into the prevention program for the disease in the defined area.

Some embodiments provide a computer system for determining a need for disease prevention programs in a defined area.

The computer system can be configured to perform the steps of: analyzing a database comprising a plurality of community health statistics; generating a plurality of geospatial data layers of incidence of disease from the plurality of community health statistics; creating a heat map comprising the plurality of geospatial data layers; segmenting the heat map into smaller defined areas; determining which of the defined areas have a high incidence of a disease; analyzing a consumer database comprising a plurality of consumer names, each of the consumer names tagged with a consumer's address, the consumer's health risk assessment, and the consumer's contact information; identifying consumers in the defined area that have an above average risk for the disease from the consumer's health risk assessment; identifying all disease prevention program providers in the defined area that are qualified to provide a prevention program for the disease; calculating a total consumer capacity of all of the prevention programs for the disease in the defined area; and comparing a total number of the consumers identified with an above average of risk for the disease to the total consumer capacity of all of the prevention programs for the disease in the defined area.

FIG. 8 illustrates a schematic block diagram of an exemplary integrator system 800, which includes an integrator application engine 810 configured to run on an integrator computer module 801.

An integrator computer module 801 comprises an integrator application engine 810 and a customer relationship management (“CRM”) system 811, which may be implemented as a software module. CRM software modules are well known to those skilled in the art. For example, a Salesforce platform (Salesforce.com, San Francisco, Calif.) can be used as the CRM 811. The integrator application engine 810 can receive 856 data from the CRM 811. Data from the integrator application engine 810 can send 855 data to populate fields in the CRM 811.

The integrator system 800 processes data from a variety of sources. The exemplary integrator 800 can access and process data from an aggregate community health database 802, consumer database 804, and optionally a socioeconomic database 806.

The aggregate community health database 802 can include data from many sources. The geospatial aggregate health database 802 can be configured as a spatial data infrastructure designed to acquire data, process it, and store results, and preserve any accompanying spatial data. The aggregate community health database 802 can include government derived geospatial health data. For example, the aggregate community health database 802 can include data from the BRFS, which is maintained by the CDC, as discussed herein. The BRFS collects data on U.S. residents regarding health-related risk behaviors, chronic health conditions, and use of preventive services. Analysis of the BRFS can yield a plurality geospatial layers, such as, for example, health-related risk behaviors, chronic health conditions, and use of preventive services.

The aggregate community health database 802 can include community health data and statistics from a state and/or a county agency. In addition, the aggregate community health database 802 can include community health data provided by a partner organization, such as a health care provider or an employer. The aggregate community health database 802 can include community health data provided by a non-profit, such as, an industry association, a patient advocacy organization, or a watchdog group. In some embodiments, the aggregate community health database 802 can include relationships between disease prevalence and social determinants of health.

In some embodiments, the aggregate community health database 802 can include licensed data 801 from a third party. For example, the licensed data 801 can include the entries of 200 different diseases and conditions from the BCBS database, which have been tagged by location.

The consumer database 804 can include data from candidates, consumers, patients, clients, participants, and the like, which can include name, contact information, email address, healthcare provider plan, and demographic data, such as, age, and ethnicity.

The consumer database 804 can include participant-specific data, which may include, for example, patient contact information, zip code, demographics, socio-economic factors, psychographics, health information, health care utilization, claims data, electronic medical record data, prescription history, and purchasing data. The consumer database 804 can include participant-specific data for the participant's health risk assessments, as well as results from a social asset survey and/or a behavioral analysis.

For example, the consumer database 804 can include participant names, which are tagged with age, weight, BMI, sex, address, email, and phone number.

In some aspects, the consumer database 804 can be created by analyzing and aggregating a large database of claims data. The consumer database 804 can include data collected by the DPP providers.

In some embodiments, the consumer database 804 can receive third-party consumer data 803. Such data 803 can be provided by an employer or a payer or a health plan. For example, an employer contracts with the integrator to provide appropriate DPPs to all employees, the employer would provide third-party consumer data 803 for all of the employees. The integrator can use 803 to contact the employees and perform a health risk assessment on each employee and may perform a social asset survey or a behavioral analysis. The results for each employee can be tagged with location and entered into the consumer database 804. In addition, the results for all the employees can be aggregated into geospatial data, which may be used by the aggregate community health database 802 as an additional dataset.

The socioeconomic database 806 can include a plurality geospatial layers for individual social determinants on a community level. The socioeconomic database 806 can include a plurality geospatial layers for each of the various economic factors. The socioeconomic database 806 can include a geospatial layers for demographics. The geospatial layers in the socioeconomic database 806 can span over an area as big as a state, or a metropolitan area, or a county. The resolution of the geospatial layers in the socioeconomic database 806 is at preferably at a zip code level or finer resolution. In some embodiments, the resolution the geospatial layers in the socioeconomic database 806 is at a census block level or at a neighborhood level.

The consumer database 804 can generate socioeconomic geospatial layers, which can be added to, or integrated with an equivalent socioeconomic geospatial layer in the socioeconomic database 806. Using socioeconomic geospatial layers generated by the consumer database 804 can increase the resolution of one or more socioeconomic geospatial layers in the socioeconomic database 806 for a particular area or location.

In some embodiments, data from the socioeconomic database 806 is included in the aggregate community health database 802.

The databases operate within the framework of HIPAA and are protected with the appropriate firewalls and other safeguards to protect and limit access to any data connected to an individual. In some configurations, the data from any of the databases can be sent thru a scrubbing program, which can be configured to clean the data, fill in missing fields, and/or eliminate duplicates. The scrubbed data can be sent to the CRM 811 software module operating within the integrator computer module 801.

After the data is received and cataloged by the CRM 811, the data provided by each of the databases are used to identify a pool of candidates in a defined location. The CRM 811 can create a plurality of geospatial layers for a heat map, which includes the data and the locations associated with different diseases and conditions.

The heat map can integrate DPP provider data, including the nature and location of local community based resources from consumer database 804, with geographical data on disease metrics from health statistics database 802 and social determinants from socioeconomic database 806 to generate the heat map. Using the heat map, the integrator can design an intervention policy for a defined location, such as, for example, for example, a zip code or a neighborhood, to improve community health in the defined location. An analysis of the heat map produces data for the defined location, which can identify, for example, the highest health risks factors by future health costs, the highest risk factors by numbers of people, the effect of social determinants on the population, and/or the percentage of the population, which has access to either medical health insurance or a government program that pays for medical costs.

An analysis of the heat map can yield the health risk factors to focus on in the defined location and the probability of reimbursement for providing a DPP to a candidate. The analysis of the heat map can provide an understanding of any social determinants, in the defined location, that may negatively affect a likelihood of a candidate successfully completing the DPP. With this understanding, social programs and/or services to overcome one or more of the social determinants can be offered to a candidate enrolling in a DPP.

For example, the CRM 811 can create heat maps for targeting a demographic subset of the population in a defined area and removing barriers to healthcare for the subset of the population.

In various embodiments, the integrator has the power to enroll people in the programs, accordingly, the integrator is uniquely qualified to combine the geospatial aggregate health data, from the aggregate community health database 802, with the individual patient profiles, from consumer database 804, to determine the demand in a defined location of a market for one or more DPPs. Based on this demand, the integrator can recruit and build out the DPP providers, in the defined location, which allows vetted participants to be enrolled in the best-fit DPP in the defined location. Since the integrator has access to the patient profile information in the consumer database 804, this allows the aggregate data to be actionable in a defined location, which can trigger can enrollment in a DPP for a candidate in the defined location.

Based on the results of a particular heat map analysis, the CRM 811 sends a message including an attached weblink or other indicia of an integrator portal, along with at least a portion of the data relating to targeted consumers, to an email distribution module 815. Email distribution modules are well known to those skilled in the art. For example, a Marketo marketing automation platform (Marketo, Inc., San Mateo, Calif.) can be used as the email distribution module. The email distribution module 815 sends emails to a plurality of targeted consumers via the cloud 817 (e.g., the internet).

Upon receipt of the email, a targeted consumer is directed to a website 825 by clicking the weblink embedded in the email. The number of targeted consumers is theoretically infinitely scalable.

A first targeted consumer 820 opens the email and clicks the weblink, which puts the first targeted consumer 820 in contact with the website 825. The first targeted consumer 820 inputs data 821 in response to a health risk assessment, a survey of personal preferences, a social asset survey, and optionally a behavioral analysis, all of which are provided through the website 825. The received data 823 from the first consumer 820 is entered into the integrator application engine 810 for analysis. The integrator application engine 810 analyzes the received data 823. Based on this analysis, the integrator application engine 810 can interface with the DPP database 827 and optionally with social/behavior database 837.

According to the received data 823, the first targeted consumer 820 has no social support needs and no behavioral support needs. Accordingly, the integrator application engine 810 can interface with the DPP database 827 designs a personalized precision DPP 852 for the first targeted consumer 820. The personalized precision DPP 852 is communicated 824 to the first targeted consumer 820. The integrator application engine 810 can enroll the first targeted consumer 820 into one or more personalized precision DPPs 852 and appropriate notices of enrollment 822 are sent to the first targeted consumer 820 and the best-fit DPP provider.

Similarly, a second targeted consumer 830 interfaces with the website 825. The second targeted consumer 830 inputs data 831 in response to a health risk assessment, a survey of personal preferences, a social asset survey, and optionally a behavioral analysis, all of which are provided through the website 825. The received data 833 from the second targeted consumer 830 is entered into the integrator application engine 810 for analysis. The integrator application engine 810 analyzes the received data 833.

According to the received data 833, the second targeted consumer 830 has social support needs but no behavioral support needs. Accordingly, the integrator application engine 810 can interface with the DPP database 827 and social database 837 and designs a second personalized precision DPP 853, which includes a social support component, for the second targeted consumer 830. The second personalized precision DPP 853, including the social support component, is communicated 834 to the second targeted consumer 830. The integrator application engine 810 can enroll the second targeted consumer 830 into the DPP program or programs associated with the second personalized precision DPP 853. The integrator application engine 810 can interface with a social support provider and set up the social support component for the second targeted consumer 830. Appropriate notices of enrollment 832 are sent to second targeted consumer 830, the best-fit DPP provider, and the social support provider.

A third targeted consumer 840 interfaces with the website 825. The third targeted consumer 840 inputs data 841 in response to a health risk assessment, a survey of personal preferences, a social asset survey, and optionally a behavioral analysis. The received data 843 from the third targeted consumer 840 is entered into the integrator application engine 810 for analysis. The integrator application engine 610 analyzes the received data 843.

According to the received data 833, the third targeted consumer 840 has no social support needs. However, third targeted consumer 840 has behavioral support needs. Accordingly, the integrator application engine 810 can interface with the DPP database 827 and social/behavioral database 837 and designs a third personalized precision DPP 854, which includes a behavioral support component. The third personalized precision DPP 854, including the behavioral component, is communicated 844 to the third targeted consumer 840. The integrator application engine 810 can enroll the third targeted consumer 840 into the third personalized precision DPP 854. The integrator application engine 810 can interface with a behavioral support provider and set up the behavioral support component for the third targeted consumer 840. Appropriate notices of enrollment 842 are sent to the third targeted consumer 840, the best-fit DPP provider, and the behavioral support provider.

In some embodiments, the integrator system 800 can be configured to automatically send a notice to eligible participants if their address is within a hot zone of a heat map for a particular chronic disease. The integrator system 800 can be configured to automatically send a notice to eligible participants if their age/demographics match those of the hot zone of a heat map for a particular chronic disease.

In a proactive approach, the integrator system 800 can be configured to automatically send a notice to an eligible participant if a change in the participant personal risk factors triggers an invitation to a DPP. For example, a participant is being monitored with a biometric device, such as, a smart scale. The integrator system 800 receives and stores BMI and other data sent from the biometric device. If the integrator system 800 determines that the participant's BMI is now in a high risk zone, the integrator computer 800 can be configured to enroll the participant into DPP and a notice of the enrollment to the participant.

In an embodiment, if the heat map analysis suggests that there are no physical locations offering a needed program and tele-health is not an attractive option, the system can augment the build out of program services using a digital file which the participant can print and/or view videos, thereby, take the class, for example, in multiple languages.

A series of algorithms and/or branched logic may be used to determine one or more “best-fit” programs for a candidate, thereby allowing the candidate to explore options based on his or her expressed preferences. Successful application of these insights can positively drive program engagement and influence health and wellness behaviors, and support ongoing retention and successful program completion.

Some embodiments provide systems and methods for providing a personalized precision prevention plan for a consumer, which may be event driven by DPP milestones that mark the progress of the patient through the DPP and initiate corresponding payments to the DPP provider. The personalized precision prevention plan can be designed to enhance and encourage the patient to have meaningful engagement with the DPP provider, which increases the probability the patient completes a DPP.

In one example, meaningful engagement contemplates the satisfaction of predetermined milestones associated with the DPP, which can then be used to trigger a milestone payment from the payer to the DPP provider. Accordingly, meaningful engagement is an event, which initiates an action. For example, upon meaningful engagement, the DPP provider is sent a payment. For example, upon meaningful engagement, a claim is sent to the payer. Meaningful engagement metrics can be tracked for a plurality of participants and a plurality of providers to improve the system. The systems and methods may also be configured to track meaningful engagement between the patient and the optimal DPP provider.

In some configurations, a community dashboard can be configured to analyze a combination geospatial data to identify needs and suggest programs to target a particular participant or group of participants. The community dashboard suggests locations to build new provider facilities to increase capacity for one or more programs. An integrator system, based on the results from the community dashboard, can be configured to confirm eligibility; enroll a participant, send a claim to and receive payment from an insurance provider, initiate participant evaluation of a program; and use the evaluations as feedback to improve community health.

Some methods can include the steps of: identifying participant's needs, suggesting programs to meet the needs, confirming participant's eligibility, enrolling the participant in one or more of the programs, receiving payment from participant's healthcare provider, receiving participant's feedback on the programs, assessing participant's level of satisfaction with the programs, and personalizing disease prevention for the participant.

Various embodiments provide a computer implemented method for using aggregate community health statistics to drive patient enrollment in disease prevention programs.

The method comprises the steps of: analyzing a database comprising a plurality of community health statistics; generating a plurality of geospatial data layers of disease incidence from the plurality of community health statistics; creating a heat map comprising the plurality of geospatial data layers; segmenting the heat map into smaller defined areas; determining which of the defined areas have a high incidence of a preventable disease; analyzing a patient database comprising a plurality of patient names, each of the patient names tagged with a patient's address and the patient's contact information; identifying a group of patients in the defined areas with the high incidence of the preventable disease; contacting each of the identified patients in the group of patients; and enrolling at least a portion of the identified patients into a disease prevention program for the preventable disease.

The method can further comprise the steps of: surveying each of the identified patients for personal preferences for the disease prevent program; and determining a best-fit disease prevention program provider for each of the identified patients based on comparing the personal preferences. The best fit disease prevention program provider can be located in the defined area.

The method can further comprise the steps of: submitting a claim for the disease prevention program for each of the identified patients to a group of payers; and receiving a payment for at least one claim from the group of payers. The method can include sending a portion of the payment to a disease prevention program provider. The payment can include a premium paid to a system facilitator.

The method can further comprise the steps of: opening an account for each of the identified patients enrolled in the disease prevention program; including a transaction fee for the account in the claim; and sending the transaction fee included in the payment to a system facilitator.

The method can further comprise the steps of: monitoring progress in the prevention program for one of the identified patients; reporting achievement of a program milestone to the payer; and receiving payment from the payer. The payer can be a health insurance provider for the one identified patient.

The method can further comprise the steps of: surveying each of the identified patients for social needs for success in the disease prevention program; determining a highest impact social need for increasing a likelihood of success in the disease prevention program for each of the identified patients; developing a best-fit social solution for the highest impact social need for each of the identified patients with a group of social support providers; determining a best-fit social support provider for each of the identified patients from the group of social support providers; and enrolling each of the identified patients in the best-fit social solution with the best-fit social support provider.

The method can further comprise the steps of: bundling a claim for the disease prevention program and a claim for the best-fit social solution for each of the identified patients; sending a bundled claim for each of the identified patients to a group of payers; and receiving a payment for one of the bundled claims from the group of payers.

The method can further comprise the steps of: sending a first portion of the payment to a disease prevention program provider; and sending a second portion of the payment to a social support provider. The method can include sending a third portion of the payment to a system facilitator.

The method can further comprise the steps of: determining which of the defined areas have a high incidence of a second preventable disease; analyzing the patient database; identifying a second group of patients in the defined areas with the high incidence of the second preventable disease; contacting each of the identified patients in the second group of patients; and enrolling at least a portion of the identified patients in the second group of patients into a disease prevention program for the second preventable disease.

Various embodiments provide a computer system for using aggregate community health statistics to drive patient enrollment in disease prevention programs.

The computer system can be configured to perform the steps of: analyzing a database comprising a plurality of community health statistics; generating a plurality of geospatial data layers of disease incidence from the plurality of community health statistics; creating a heat map comprising the plurality of geospatial data layers; segmenting the heat map into smaller defined areas; determining which of the defined areas have a high incidence of a preventable disease; analyzing a patient database comprising a plurality of patient names, each of the patient names tagged with a patient's address and the patient's contact information; identifying a group of patients in the defined areas with the high incidence of the preventable disease; contacting each of the identified patients in the group of patients; and enrolling at least a portion of the identified patients into a disease prevention program for the preventable disease.

The computer system can be further configured to perform the steps of: surveying each of the identified patients for personal preferences for the disease prevent program; and determining a best-fit disease prevention program provider for each of the identified patients based on comparing the personal preferences.

The computer system can be further configured to perform the steps of: submitting a claim for the disease prevention program for each of the identified patients to a group of payers; receiving a payment for at least one claim from the group of payer; and sending a portion of the payment to a disease prevention program provider.

The computer system can be further configured to perform the steps of: determining which of the defined areas have a high incidence of a second preventable disease; analyzing the patient database; identifying a second group of patients in the defined areas with the high incidence of the second preventable disease; contacting each of the identified patients in the second group of patients; and enrolling at least a portion of the identified patients in the second group of patients into a disease prevention program for the second preventable disease.

The computer system can be further configured to perform the steps of: surveying each of the identified patients for social needs for success in the disease prevention program; determining a highest impact social need for increasing a likelihood of success in the disease prevention program for each of the identified patients; developing a best-fit social solution for the highest impact social need for each of the identified patients with a group of social support providers; determining a best-fit social support provider for each of the identified patients from the group of social support providers; and enrolling each of the identified patients in the best-fit social solution with the best-fit social support provider.

Various embodiments provide computer code stored in a non-transient medium for performing, when executed by a computer processor, the steps of: analyzing a database comprising a plurality of community health statistics; generating a plurality of geospatial data layers of disease incidence from the plurality of community health statistics; creating a heat map comprising the plurality of geospatial data layers; segmenting the heat map into smaller defined areas; determining which of the defined areas have a high incidence of a preventable disease; analyzing a patient database comprising a plurality of patient names, each of the patient names tagged with a patient's address and the patient's contact information; identifying a group of patients in the defined areas with the high incidence of the preventable disease; contacting each of the identified patients in the group of patients; and enrolling at least a portion of the identified patients into a disease prevention program for the preventable disease.

Those skilled in the art will appreciate that the systems and methods described herein may contemplate any prevention or treatment program, as well as chronic disease management, telemedicine, medication and dosage adherence, social services, behavioral health, and the like.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

What is claimed:

1. A computer implemented method for using aggregate community health statistics to drive patient enrollment in disease prevention programs, the method comprising:
Analyzing, with a processor, a database comprising a plurality of community health statistics;
generating a plurality of geospatial data layers of disease incidence from the plurality of community health statistics;
creating a heat map associated with the plurality of geospatial data layers;
segmenting the heat map into smaller defined areas;
determining which of the defined areas have a high incidence of a preventable disease;
analyzing a patient database comprising a plurality of patient names, each of the patient names tagged with a patient's address and the patient's contact information;
identifying a group of patients in the defined areas with the high incidence of the preventable disease;
contacting each of the identified patients in the group of patients; and
enrolling at least a portion of the identified patients into a disease prevention program for the preventable disease.

2. The method according to claim 1, further comprising:
surveying each of the identified patients for personal preferences for the disease prevent program; and
determining a best-fit disease prevention program provider for each of the identified patients based on comparing the personal preferences.

3. The method according to claim 2, wherein the best fit disease prevention program provider is located in the defined area.

4. The method according to claim 1, further comprising:
submitting a claim for the disease prevention program for each of the identified patients to a group of payers; and
receiving a payment for at least one claim from the group of payers.

5. The method according to claim 4, further comprising:
sending a portion of the payment to a disease prevention program provider.

6. The method according to claim 4, wherein the payment includes a premium paid to a system facilitator.

7. The method according to claim 4, further comprising:
opening an account for each of the identified patients enrolled in the disease prevention program;
including a transaction fee for the account in the claim; and
sending the transaction fee included in the payment to a system facilitator.

8. The method according to claim 1, further comprising:
monitoring progress in the prevention program for one of the identified patients;
reporting achievement of a program milestone to the payer; and
receiving payment from the payer.

9. The method according to claim 8, wherein the payer is a health insurance provider for the one identified patient.

10. The method according to claim 1, further comprising:
surveying each of the identified patients for social needs for success in the disease prevention program;
determining a highest impact social need for increasing a likelihood of success in the disease prevention program for each of the identified patients;
developing a best-fit social solution for the highest impact social need for each of the identified patients with a group of social support providers;
determining a best-fit social support provider for each of the identified patients from the group of social support providers; and
enrolling each of the identified patients in the best-fit social solution with the best-fit social support provider.

11. The method according to claim 10, further comprising:
bundling a claim for the disease prevention program and a claim for the best-fit social solution for each of the identified patients;
sending a bundled claim for each of the identified patients to a group of payers; and
receiving a payment for one of the bundled claims from the group of payers.

12. The method according to claim ii, further comprising:
sending a first portion of the payment to a disease prevention program provider; and
sending a second portion of the payment to a social support provider.

13. The method according to claim 12, further comprising:
sending a third portion of the payment to a system facilitator.

14. The method according to claim 1, further comprising:
determining which of the defined areas have a high incidence of a second preventable disease;
analyzing the patient database;
identifying a second group of patients in the defined areas with the high incidence of the second preventable disease;
contacting each of the identified patients in the second group of patients; and
enrolling at least a portion of the identified patients in the second group of patients into a disease prevention program for the second preventable disease.

15. A computer system for using aggregate community health statistics to drive patient enrollment in disease prevention programs, the computer system configured to perform the steps of:
analyzing a database comprising a plurality of community health statistics;
generating a plurality of geospatial data layers of disease incidence from the plurality of community health statistics;
creating a heat map comprising the plurality of geospatial data layers;
segmenting the heat map into smaller defined areas;
determining which of the defined areas have a high incidence of a preventable disease;
analyzing a patient database comprising a plurality of patient names, each of the patient names tagged with a patient's address and the patient's contact information;
identifying a group of patients in the defined areas with the high incidence of the preventable disease;
contacting each of the identified patients in the group of patients; and
enrolling at least a portion of the identified patients into a disease prevention program for the preventable disease.

16. The computer system according to claim 15, further configured to perform the steps of:
surveying each of the identified patients for personal preferences for the disease prevent program; and
determining a best-fit disease prevention program provider for each of the identified patients based on comparing the personal preferences.

17. The computer system according to claim 15, further configured to perform the steps of:
submitting a claim for the disease prevention program for each of the identified patients to a group of payers;
receiving a payment for at least one claim from the group of payer; and
sending a portion of the payment to a disease prevention program provider.

18. The computer system according to claim 15, further configured to perform the steps of:
determining which of the defined areas have a high incidence of a second preventable disease;
analyzing the patient database;
identifying a second group of patients in the defined areas with the high incidence of the second preventable disease;
contacting each of the identified patients in the second group of patients; and
enrolling at least a portion of the identified patients in the second group of patients into a disease prevention program for the second preventable disease.

19. The computer system according to claim 15, further configured to perform the steps of:
surveying each of the identified patients for social needs for success in the disease prevention program;
determining a highest impact social need for increasing a likelihood of success in the disease prevention program for each of the identified patients;
developing a best-fit social solution for the highest impact social need for each of the identified patients with a group of social support providers;
determining a best-fit social support provider for each of the identified patients from the group of social support providers; and
enrolling each of the identified patients in the best-fit social solution with the best-fit social support provider.

20. Computer code stored in a non-transient medium for performing, when executed by a computer processor, the steps of:
analyzing a database comprising a plurality of community health statistics;
generating a plurality of geospatial data layers of disease incidence from the plurality of community health statistics;
creating a heat map comprising the plurality of geospatial data layers;
segmenting the heat map into smaller defined areas;
determining which of the defined areas have a high incidence of a preventable disease;
analyzing a patient database comprising a plurality of patient names, each of the patient names tagged with a patient's address and the patient's contact information;
identifying a group of patients in the defined areas with the high incidence of the preventable disease;
contacting each of the identified patients in the group of patients; and
enrolling at least a portion of the identified patients into a disease prevention program for the preventable disease.
